SEQUENCE LISTING (9 sequences)

SEQ ID NO: 1 - protein, 11 aa, Artificial Sequence
  synthetic
  MISC_FEATURE (2)..(2): Xaa = Ala, Arg, Asn, Asp, Gln, Glu, Gly, His, Ile, Leu, Lys, Met, Phe, Pro, pyrrolysine, selenocysteine, Ser, Thr, Trp, Tyr or Val.
  MISC_FEATURE (3)..(10): any one or all of amino acids 3 to 10 can be present or absent; Xaa = Ala, Arg, Asn, Asp, Gln, Glu, Gly, His, Ile, Leu, Lys, Met, Phe, Pro, pyrrolysine, selenocysteine, Ser, Thr, Trp, Tyr or Val.
CXXXXXXXXXC

SEQ ID NO: 2 - protein, 8 aa, Artificial Sequence
  synthetic
  MISC_FEATURE (1)..(1): Xaa = Ala, Arg, Asn, Asp, Gln, Glu, Gly, His, Ile, Leu, Lys, Met, Phe, Pro, pyrrolysine, selenocysteine, Ser, Thr, Trp, Tyr or Val.
  MISC_FEATURE (5)..(7): Xaa = Ala, Arg, Asn, Asp, Gln, Glu, Gly, His, Ile, Leu, Lys, Met, Phe, Pro, pyrrolysine, selenocysteine, Ser, Thr, Trp, Tyr or Val.
  misc_feature (8)..(8): Xaa can be any naturally occurring amino acid
XCPDCXXX

SEQ ID NO: 3 - protein, 4 aa, Artificial Sequence
  synthetic
CPDC

SEQ ID NO: 4 - DNA, 95 bp, Artificial Sequence
  synthetic
  misc_feature (22)..(23): n = A, T, G, or C
  misc_feature (24)..(24): m = A or C
  misc_feature (25)..(26): n = A, T, G, or C
  misc_feature (27)..(27): m = A or C
  misc_feature (28)..(29): n = A, T, G, or C
  misc_feature (42)..(42): m = A or C
  misc_feature (43)..(44): n = A, T, G, or C
aaggccgcttttgcgggatccnnmnnmnngcagtccgggcamnntgcagcgaaagacagcatcggaacgagggtagcaacggctacagaggcttt

SEQ ID NO: 5 - DNA, 89 bp, Artificial Sequence
  synthetic
ttaatggaaacttcctcatgaaaaagtctttagtcctcaaagcctctgtagccgttgctaccctcgttccgatgctgtctttcgctgca

SEQ ID NO: 6 - DNA, 30 bp, Artificial Sequence
  synthetic
cctattggttaaaaaatgagctgatttaac

SEQ ID NO: 7 - DNA, 19 bp, Artificial Sequence
  synthetic
ccgaaatcggcaaaatccc

SEQ ID NO: 8 - DNA, 19 bp, Artificial Sequence
  synthetic
gggattttgccgatttcgg

SEQ ID NO: 9 - DNA, 30 bp, Artificial Sequence
  synthetic
gttaaatcagctcattttttaaccaatagg

(19) United States
(12) Patent Application Publication
(10) Pub. No.: US 2023/0304025 A1
(43) Pub. Date: Sep. 28, 2023

(54) M13 BACTERIOPHAGE WITH A HIGH CYSTEINE CONTENT AND GENETICALLY ENGINEERABLE HYDROGELS

(71) Applicant: Massachusetts Institute of Technology, Cambridge, MA (US)

(72) Inventors: Angela Belcher, Lexington, MA (US); William Records, Cambridge, MA (US); Nimrod Heldman, Cambridge, MA (US)

(73) Assignee: Massachusetts Institute of Technology, Cambridge, MA (US)

(21) Appl. No.: 18/017,139

(22) PCT Filed: Jul. 20, 2021
(86) PCT No.: PCT/US2021/042418

§ 371 Date: Jan. 20, 2023

Related U.S. Application Data
(60) Provisional application No. 63/054,215, filed on Jul. 20, 2020.

(51) Int. Cl. C12N 15/70; C12N 1/20; C12N 11/14; C12N 9/22

(52) U.S. Cl. CPC C12N 15/70; C12N 1/20; C12N 9/22; C12N 11/14; C12N 2795/14022; C12N 2795/14043

(57) ABSTRACT

A genetically engineered bacteriophage that can be crosslinked to form a solid material, for example, a hydrogel.

### CROSS REFERENCE TO RELATED APPLICATIONS

This application claims priority to and the benefit of U.S. Provisional Pat. Application No. 63/054,215 filed Jul. 20, 2020, which is incorporated herein by reference in its entirety.

### STATEMENT OF FEDERAL GOVERNMENT SUPPORT

This invention was made with Government support under Grant No. HR0011-15-C-0084 awarded by the Defense Advanced Research Projects Agency (DARPA). The Government has certain rights in the invention.

### SEQUENCE LISTING

This application incorporates by reference in its entirety the Sequence Listing entitled “270721-MIT22369WO-491108_ST25.txt” (3,541 bytes), which was created on Jul. 13, 2021, and filed electronically herewith.

### TECHNICAL FIELD

This invention relates to an engineered bacteriophage, solid materials made from engineered bacteriophage and method of making the engineered bacteriophage and solid materials.

### BACKGROUND

Controlled synthesis of solid materials can be important for a number of applications.

### SUMMARY

In one aspect, a genetically engineered bacteriophage can include a plurality of peptides expressed at a surface of the bacteriophage, each peptide can include two or more cysteine residues.

In another aspect, a method of forming a solid material can include providing a plurality of genetically engineered bacteriophages, each genetically engineered bacteriophage comprising a plurality of peptides expressed at a surface of the bacteriophage, each peptide having two or more cysteine residues, and crosslinking the plurality of genetically engineered bacteriophages.

In another aspect, a method of making a genetically engineered bacteriophage can include expressing a plurality of peptides at a surface of the bacteriophage, each peptide having two or more cysteine residues.

In another aspect, a method of making a genetically engineered bacteriophage genome construct can include contacting a bacteriophage genome with restriction enzymes to produce a cleaved linear bacteriophage genome, contacting a heterologous nucleic acid with a second set of restriction enzymes to produce a cleaved heterologous nucleic acid, the heterologous nucleic acid can encode a peptide having two or more cysteine residues, and ligating the cleaved linear bacteriophage genome with the heterologous nucleic acid in the presence of a ligase enzyme to produce a genetically engineered bacteriophage genome construct.

In another aspect, a method of making a genetically engineered bacteriophage genome construct, can include contacting a bacteriophage genome with a DNA polymerase enzyme in a polymerase chain reaction (PCR) to produce an extended bacteriophage genome; and ligating the extended bacteriophage genome with a heterologous nucleic acid in the presence of a ligase enzyme to produce a genetically engineered bacteriophage genome, wherein the heterologous nucleic acid encodes a peptide having two or more cysteine residues. Optionally, the method can include first contacting a bacteriophage genome with restriction enzymes to produce a cleaved linear bacteriophage genome.

In another aspect, a method of making a genetically engineered bacteriophage genome construct can include contacting a bacteriophage genome with DNA polymerase enzyme in a polymerase chain reaction (PCR), the enzyme amplifies a part of the bacteriophage genome, contacting a heterologous nucleic acid, the heterologous nucleic acid encoding a peptide having two or more cysteine residues, with DNA polymerase enzyme in a different PCR to amplify the heterologous nucleic acid, or contacting two complementary heterologous nucleic acids, one of the heterologous nucleic acids encoding a peptide having two or more cysteine residues, with DNA polymerase enzyme in a different PCR to amplify and anneal the two complementary nucleic acids; and ligating the amplified bacteriophage genome with the amplified heterologous nucleic acid in the presence of an exonuclease, a DNA polymerase, and a DNA ligase to produce a genetically engineered bacteriophage genome construct.

In another aspect, a conjugate can include the genetically engineered bacteriophage described herein.

In another aspect, a solid material can include the genetically engineered bacteriophage described herein. For example, the solid material can be a hydrogel.

In another aspect, a composition can include a plurality of polynucleotides encoding the plurality of peptides expressed at the surface of the bacteriophage. In another aspect, a construct can include the polynucleotides operably linked to a promoter and/or an operator.

In another aspect, a pharmaceutical composition can include the genetically engineered bacteriophage described herein and a pharmaceutically acceptable excipient.

In another aspect, a method for treating a disease or medical condition in a subject in need thereof can include administering the pharmaceutical compositions described herein.

In another aspect, a method of synthesizing inorganic materials is disclosed herein. In certain circumstances, the method of synthesizing inorganic materials can be achieved by producing a solid material including the genetically engineered bacteriophage described herein and contacting the inorganic material with the solid material including the genetically engineered bacteriophage or contacting the inorganic material with the genetically engineered bacteriophage prior to the production of the solid material.

In another aspect, a method for detecting analytes in an entity is disclosed herein. In certain circumstances, the method can be employed by contacting a moiety capable of binding or recognizing the entity to be analyzed with the genetically engineered bacteriophage disclosed herein, producing a solid material including the modified bacteriophage, contacting the entity or a sample of the entity with the solid material including the modified bacteriophage, and detecting a change in the solid material and/or the moiety capable of binding or recognizing the entity.

In another aspect, a method for detecting analytes in an entity is disclosed herein. In certain circumstances, the method can be employed by expressing a moiety capable of binding or recognizing the entity to be analyzed at the surface of the genetically engineered bacteriophage disclosed herein, producing a solid material including the modified bacteriophage, and contacting the entity or a sample of the entity with the solid material including the modified bacteriophage.

In another aspect, a method for detecting analytes in an entity is disclosed herein. In certain circumstances, the method can be employed by expressing a moiety capable of binding or recognizing the entity to be analyzed at the surface of the genetically engineered bacteriophage disclosed herein, contacting the entity or a sample of the entity with the solid material including the modified bacteriophage, and producing a solid material including the modified bacteriophage.

In certain circumstances, the method of detecting can include a detecting step, as described herein. For example, a hydrogel can be formed prior to contacting the entity to be analyzed and a measurable change in the system can occur in the presence of the analyte.

In certain circumstances, detecting can be one or more of: (a) detecting a change in fluorescence or absorbance when the analyte is added of the solid material and/or the moiety capable of binding or recognizing the entity; (b) detecting is a phase change of the solid material upon contacting the analyte, wherein the phase change is a phase change to a liquid, fluid, and/or gas; or (c) detecting is a change in the solid material and/or the moiety capable of binding or recognizing the entity upon contacting the analyte, wherein the change is a change in surface plasmon resonance, temperature or pH, or wherein the change is the solid material and/or the moiety capable of binding or recognizing the entity becoming surface plasmon resonance active.

In another aspect, a method of producing a solid material is disclosed herein. In certain circumstances, the method can be employed by conjugating at least one molecule onto the surface of the genetically engineered bacteriophage described herein to produce a conjugated bacteriophage, and producing a solid material including the conjugated bacteriophage.

In another aspect, a genetically engineered bacteriophage can have (i) a first plurality of peptides expressed at a surface of the bacteriophage, wherein each peptide includes two or more cysteine residues and (ii-1) a second plurality of peptides expressed at a surface of the bacteriophage, wherein the second plurality of peptides includes a fused peptide or (ii-2) a second plurality of peptides, wherein the second plurality of peptides comprises peptides that are expressed and/or secreted by a bacterial host cell is described herein. In certain circumstances, each peptide of the (ii) second plurality of peptides can have two or more cysteine residues. In certain circumstances, each peptide of the (ii) second plurality of peptides cannot have two or more cysteine residues.

In another aspect, a method of making a genetically engineered bacteriophage can have (i) a first plurality of peptides at a surface of the bacteriophage, wherein each peptide includes two or more cysteine residues and (ii-1) a second plurality of peptides expressed at a surface of the bacteriophage, wherein the second plurality of peptides includes a fused peptide or (ii-2) a second plurality of peptides, wherein the second plurality of peptides comprises peptides that are expressed and/or secreted by a bacterial host cell is described herein.

In another aspect, a conjugate can include the genetically engineered bacteriophage having a first and a second plurality of peptides described herein.

In another aspect, a solid material can include the genetically engineered bacteriophage having a first and a second plurality of peptides described herein. For example, the solid material can be a hydrogel.

In another aspect, a method of making a genetically engineered bacteriophage can include expressing a first plurality of peptides at a surface of the bacteriophage, each peptide having two or more cysteine residues and a second plurality of peptides that are or are not expressed at a surface of the bacteriophage.

In another aspect, a method of making a genetically engineered bacteriophage genome construct can include contacting a bacteriophage genome with restriction enzymes to produce a cleaved linear bacteriophage genome, contacting a heterologous nucleic acid with a second set of restriction enzymes to produce a cleaved heterologous nucleic acid, the heterologous nucleic acid selected from (ii-1) or (ii-2), and ligating the cleaved linear bacteriophage genome with the heterologous nucleic acid in the presence of a ligase enzyme to produce a genetically engineered bacteriophage genome construct. In certain circumstances, heterologous nucleic acid (ii-1) can encode a pVIII major coat protein or a pIII, pVI, or pIX minor coat protein fused to a distinct moiety designed for a specific purpose and heterologous nucleic acid (ii-2) can encode a plurality of peptides expressed and/or secreted by a bacterial host cell when the bacteriophage genome construct is propagated therein.

In another aspect, a method of making a genetically engineered bacteriophage genome construct, can include contacting a bacteriophage genome with a DNA polymerase enzyme in a polymerase chain reaction (PCR) to produce an extended bacteriophage genome; and ligating the extended bacteriophage genome with a first and a second heterologous nucleic acid selected from (i) and (ii) in the presence of a ligase enzyme to produce a genetically engineered bacteriophage genome construct. Optionally, the method can include first contacting a bacteriophage genome with restriction enzymes to produce a cleaved linear bacteriophage genome. In certain circumstances, heterologous nucleic acid (ii-1) can encode a pVIII major coat protein or a pIII, pVI, pVII, or pIX minor coat protein fused to a distinct moiety designed for a specific purpose and heterologous nucleic acid (ii-2) can encode a plurality of peptides expressed and/or secreted by a bacterial host cell when the bacteriophage genome construct is propagated therein.

In another aspect, a method of making a genetically engineered bacteriophage genome construct can include contacting a bacteriophage genome with DNA polymerase enzyme in a polymerase chain reaction (PCR), the enzyme amplifies a part of the bacteriophage genome, contacting a first and a second heterologous nucleic acid, the heterologous nucleic acid selected from (i) and (ii), with DNA polymerase enzyme in a different PCR to amplify the heterologous nucleic acid, or contacting two complementary heterologous nucleic acids, one of the heterologous nucleic acids encoding a peptide having two or more cysteine residues, with DNA polymerase enzyme in a different PCR to amplify and anneal the two complementary nucleic acids; and ligating the amplified bacteriophage genome with the amplified heterologous nucleic acid in the presence of an exonuclease, a DNA polymerase, and a DNA ligase to produce a genetically engineered bacteriophage genome construct. In certain circumstances, heterologous nucleic acid (ii-1) can encode a pVIII major coat protein or a pIII, pVI, pVII, or pIX minor coat protein fused to a distinct moiety designed for a specific purpose and heterologous nucleic acid (ii-2) can encode a plurality of peptides expressed and/or secreted by a bacterial host cell when the bacteriophage genome construct is propagated therein.

In another aspect, a composition can include a first plurality of polynucleotides encoding the first plurality of peptides and a second plurality of polynucleotides encoding the second plurality of peptides described herein.

In another aspect, a construct can include the first and second polynucleotides described herein, and the first and/or second plurality of polynucleotides can be operably linked to a promoter and/or an operator.

In certain circumstances, the two or more cysteine residues can form intra-peptide disulfide bonds within each peptide.

In certain circumstances, the plurality of peptides can be displayed proximate to the N terminus of a plurality of pVIII major coat proteins.

In certain circumstances, each peptide can include a pVIII major coat protein.

In certain circumstances, each peptide can include a Cys-Xaa-(Xaa)ₙ-Cys (CX(X)ₙC) (SEQ ID NO: 1) motif, where n is 1, 2, 3, 4, 5, 6, 7, or 8.

In certain circumstances, each peptide can be a CX(X)ₙC (SEQ ID NO: 1) motif, wherein n is 1.

In certain circumstances, the CXXC (SEQ ID NO: 1) motif can be included in a XCPDCXXX (SEQ ID NO: 2) sequence.

In certain circumstances, each X or Xaa can be any of the twenty natural amino acids, pyrrolysine, selenocysteine, or a synthetic amino acid.

In certain circumstances, the bacteriophage can be an M13, fd, f1, or ZJ/2 (Ff type) filamentous bacteriophage.

In certain circumstances, each peptide can be glycosylated.

In certain circumstances, each peptide can further comprise at least one protease-cleavable amino acid sequence distal to the two or more cysteine residues.

In certain circumstances, the peptides in the plurality of peptides are structurally substantially the same or have a similar function.

In certain circumstances, the peptides in the plurality of peptides include two or more structurally and/or functionally distinct populations of peptides. In certain circumstances, the two or more structurally and/or functionally distinct populations of peptides function in tandem, sequentially, or in a cascade.

In certain circumstances, the bacteriophage can further include at least one of a targeting moiety, an antibody, an antibody fragment, a bi-specific T-cell engager, an affibody, a nanobody, a cell penetrating peptide, a cytokine, a growth factor, a DNA repair enzyme, an opioid receptor-binding peptide, a protease, or a hormone.

In certain circumstances, the at least one antibody is anti-PD-1, anti-PD-L1, or anti-CTLA4; the at least one antibody fragment is a single-chain variable antibody fragment; the at least one cytokine is IL-2, IL-7, IL-18, or IL-27; the at least one growth factor is IGF, NGF, GDNF, FGF, VEGF, TGF-alpha fragment, TGF-beta fragment, PDGF, or macrophage activator; the at least one DNA repair enzyme is endonuclease V; the at least one opioid receptor-binding peptide is enkephalin or substance P; and/or the at least one hormone is insulin, glucagon, ghrelin, angiotensin, or thyroid-stimulating hormone (TSH).

In certain circumstances, the bacteriophage can further include at least one of a biotinylation protein, an antibiotic resistance gene, a bioluminescent protein, a fluorescent protein, or a chemiluminescent protein. In certain circumstances, the biotinylation protein is BirA; the bioluminescent protein is aequorin, firefly luciferase, Renilla luciferase, red luciferase, or nanoluciferase; the fluorescent protein is EGFP, EYFP, ECFP, superfolder GFP, dsRed, mCherry, mOrange, mOrange2, mRaspberry, mTangerine, mApple, mRuby, mPlum, mKate1, mKate2, mKO2, mNeptune, mNeptune681, mNeptune684, mTurquoise, TagBFP, TagRFP675, azurite, EBFP2, mKalama1, iRFP682, iRFP713, iRFP720, miRFP703, miRFP670, miRFP670nano, miRFP682, miRFP702, miRFP703, miRFP709, miRFP713, miRFP720, iBlueberry, Wi-Phy, or mIFP; and/or the chemiluminescent protein is β-galactosidase, alkaline phosphatase, or horseradish peroxidase (HRP).

In certain circumstances, the bacteriophage can further comprises at least one different plurality of peptides expressed at the surface of the bacteriophage. In certain circumstances, the at least one different plurality of peptides is displayed proximate to the N terminus of a plurality of pIII minor coat proteins; proximate to the N terminus of a plurality of pIX minor coat proteins; proximate to the N terminus of a plurality of pVI minor coat proteins; and/or proximate to the N terminus of a plurality of pVII minor coat proteins.

In certain circumstances, the at least one different plurality of peptides displayed proximate to the N terminus of a plurality of a pIII, pIX, pVI, or pVII minor coat protein is substantially the same as, or has a similar function to, the at least one different plurality of peptides displayed proximate to the N terminus of a plurality of a different pIII, pIX, pVI, or pVII minor coat protein.

In certain circumstances, the at least one different plurality of peptides displayed proximate to the N terminus of a plurality of pIII minor coat proteins is substantially the same as or has a similar function to the at least one different plurality of peptides displayed proximate to the N terminus of a plurality of pIX, pVI, and/or pVII minor coat proteins.

In certain circumstances, the at least one different plurality of peptides displayed proximate to the N terminus of a plurality of a pIII, pIX, pVI, or pVII minor coat protein is different as or has a different function from at least one protein or peptide displayed proximate to the N terminus of a plurality of a different pIII, pIX, pVI, or pVII minor coat protein.

In certain circumstances, the at least one different plurality of peptides displayed proximate to the N terminus of a plurality of pIII minor coat proteins is different or has a different function from the at least one different plurality of peptides displayed proximate to the N terminus of a plurality of pIX, pVI, and/or pVII minor coat proteins.

In certain circumstances, the polynucleotide can be operably linked to a promoter.

In certain circumstances, the promoter can be an inducible promoter or a constitutive promoter.

In certain circumstances, the promoter can be an inducible promoter.

In certain circumstances, the inducible promoter can be induced by isopropyl β-D-thiogalactopyranoside (IPTG) or light.

In certain circumstances, the solid material can include a disulfide crosslinking agent crosslinking the plurality of genetically engineered bacteriophage. In certain circumstances, the disulfide crosslinking agent is norbornene, maleimide, an alkene, an acrylate, or a functional derivative thereof.

In certain circumstances, prior to crosslinking, at least one thiol radical and/or at least one carbon radical is formed in the plurality of the genetically engineered bacteriophage, wherein the at least one thiol and/or carbon radical reacts with a second thiol and/or carbon radical to crosslink the plurality of the genetically engineered bacteriophage.

In certain circumstances, the crosslinking can include irradiating with light or heating the plurality of the bacteriophage.

In certain circumstances, crosslinking can further include adding a photoinitiator, a chemical free radical initiator and/or a thermal free radical initiator. In certain circumstances, chemical free radical initiator is ammonium persulfate, hydrogen peroxide, organic peroxide, or N,N,N′,N′-tetramethylethylene-1,2-diamine (TEMED). In certain circumstances, the thermal free radical initiator is 4,4-azobis(4-cyanovaleric acid) or potassium persulfate.

In certain circumstances, crosslinking does not comprise adding a photoinitiator.

In certain circumstances, crosslinking can include adding a reduction agent, a disulfide crosslinking agent, or a disulfide crosslinking agent and a reduction agent. In certain circumstances, the reduction agent is tris(2-carboxyethyl)phosphine (TCEP), tris(hydroxypropyl)phosphine (THP), or dithiothreitol (DTT). The reduction agent or disulfide crosslinking agent can be added on its own, with or without the photoinitiator or free radical initiator described above.

In certain circumstances, crosslinking is catalyzed by copper metal. In certain circumstances, the copper metal is a copper needle. In certain circumstances, the copper needle is compatible with an endoscopic device; is compatible with three-dimensional (3D) printing; and/or is compatible with 3D printing in situ (e.g., inside a body cavity).

In certain circumstances, the crosslinking can include disulfide reshuffling from an intra-peptide disulfide to an inter-peptide disulfide.

In certain circumstances, the crosslinking can include reduction followed by disulfide reshuffling.

In certain circumstances, the crosslinking can include at least one of the two or more cysteine residues to form at least one inter-peptide disulfide bond with a cysteine residue in at least one different bacteriophage after crosslinking.

In certain circumstances, crosslinking can further include formation of at least one carbon-sulfur bond.

In certain circumstances, the method can include adding a reduction agent to crosslink the plurality of genetically engineered bacteriophages.

In certain circumstances, the method can further include adding a disulfide crosslinking agent to crosslink the plurality of genetically engineered bacteriophages.

In certain circumstances, the two or more cysteine residues can form intra-peptide disulfides within each peptide prior to crosslinking.

In certain circumstances, the two or more cysteine residues can form inter-peptide disulfides between peptides of two different bacteriophages after crosslinking.

In certain circumstances, the constructs produced by the methods described herein can be propagated in at least one bacterial host cell to produce genetically engineered bacteriophages. In certain circumstances, the bacterial host cell can be a natural bacterial cell or a non-natural bacterial cell.

In certain circumstances, the plurality of genetically engineered bacteriophage can be isolated from the bacterial host cell.

In certain circumstances, the entity is a live subject (e.g., human, animal, or plant) or an immaterial object. In certain circumstances, the analyte is a pathogen and/or a substance foreign to the entity.

In certain circumstances, the molecule conjugated to the bacteriophage can be fluorophores, catalysts, polymers, polysaccharides, DNA, surfactants, positively charged molecules (e.g., methyl ammonium containing molecules), amphiphilic molecules, anti-microbial glycans, or Toll-Like Receptor agonists.

In certain circumstances, the pVIII major coat protein of the first plurality of peptides can have a different sequence or can be structurally different compared to the sequence or structure of the pVIII major coat protein of the second plurality of peptides.

In certain circumstances, the pVIII major coat protein of the first plurality of peptides can have the same or can have a similar sequence or structure compared to the sequence or structure of the pVIII major coat protein of the second plurality of peptides.

In certain circumstances, the fusion peptide designed for a specific purpose can be a peptide sequence known to have a therapeutic effect, a peptide sequence having scaffold-like properties, or a peptide sequence designed to have a reactive site for materials synthesis.

In certain circumstances, the peptide designed for a specific purpose of the (ii) second plurality of peptides can be a targeting moiety, an antibody, an antibody fragment, a bi-specific T-cell engager, an affibody, a nanobody, a cell penetrating peptide, a cytokine, a growth factor, a DNA repair enzyme, an opioid receptor-binding peptide, a protease, a hormone, a biotinylation protein, an antibiotic resistance gene, a bioluminescent protein, a fluorescent protein, or a chemiluminescent protein.

Other aspects, embodiments, and features as disclosed herein will be apparent from the following description, the drawings, and the claims.

### BRIEF DESCRIPTION OF THE DRAWINGS

FIG. 1 is a schematic drawing showing the formation of a solid network from a plurality of units including surface disulfides.

FIG. 2 is a schematic drawing showing the formation of a solid network from a plurality of genetically engineered bacteriophages by rearranging surface disulfides.

FIGS. 3A-3D are photographs showing large-area, bacteriophage-templated copper (Cu) nanofoams, prepared using photopolymerization with TPO. Crosslinked gel (FIG. 3A) before and (FIG. 3B) after Cu metallization. FIG. 3C shows an image of a crosslinked gel sandwiched between two glass substrates. FIG. 3D shows a side view of a bacteriophage-templated Cu nanofoam on glass. All scale bars are 1 cm.

FIGS. 4A-4D are photographs showing SEM characterization of large-area, bacteriophage-templated Cu nanofoams, prepared using photopolymerization with TPO. Using this light-based crosslinking technique yielded a homogeneous thin film across a number of length scales. Scale bars are 100 µm, 5 µm, 1 µm, and 500 nm in FIGS. 4A-4D, respectively.

FIGS. 5A-5D are a series of confocal and light microscope images of 3D printed structures made out of cross-linked M13 bacteriophage. FIG. 5A is a pillar array (scale bar 200 µm); FIG. 5B is a square grid (scale bar 200 µm); FIG. 5C is a honeycomb (scale bar 1.5 mm); FIG. 5D is an ear (scale bar 1.25 mm).

FIGS. 6A-6C are a series of light microscope images of materials made of 3D printed bacteriophage. FIG. 6A is a hexagon vanadium nitride (scale bar 1.6 mm); FIG. 6B is a honeycomb lead methylammonium bromide perovskite (scale bar 1.6 mm); FIG. 6C is a honeycomb lead methylammonium bromide perovskite fluorescence when excited at 500 nm (scale bar 1.6 mm).

FIGS. 7A-7C are a series of photographs and SEM of 3D printed bacteriophage. FIG. 7A shows bacteriophage 3D printed into a high surface area pillar array coated with copper by electroless deposition. The substrate is a titanium foil. (scale bar 0.4 cm); FIG. 7B is an SEM image of 3D printed pattern (scale bar 150 µm); FIG. 7C zooms on the SEM image showing the architecture of phage coated copper wires making up the 3D printed structure (scale bar 600 nm).

FIG. 8 is an exemplary schematic of different therapeutic proteins to be expressed as fusions to three different phage coat proteins.

### DETAILED DESCRIPTION

It is to be appreciated that certain aspects, modes, embodiments, variations, and features of the present methods and compositions are described below in various levels of detail in order to provide a substantial understanding of the present disclosure.

The present disclosure provides methods for integrating at least one heterologous nucleic acid sequence into a bacteriophage genome, producing genetically engineered bacteriophage that contain the at least one heterologous nucleic acid sequence, where the at least one heterologous nucleic acid sequence encodes at least one peptide having two or more cysteine residues that is expressed at a surface of the bacteriophage, and compositions thereof. The present disclosure also provides methods for using the genetically engineered bacteriophage and compositions thereof to facilitate the formation of conjugates or crosslinked solid materials, e.g., nanofoams or hydrogels. The present disclosure also relates to methods of treating disease (e.g., bacterial infection, cancer, or inflammatory condition, e.g., autoimmune or autoinflammatory disease) by administering a therapeutically effective amount of formulations or compositions comprising the genetically engineered bacteriophages as disclosed herein to a subject in need thereof.

The genetically engineered bacteriophages as disclosed herein include at least two (e.g., 2, 3, 4, 5 or more) cysteine residues on at least a plurality of peptides expressed at a surface of the bacteriophage for the purpose of forming disulfide bonds. The genetically engineered bacteriophages as disclosed herein can include at least two (e.g., 2, 3, 4, 5 or more) cysteine residues on at least a plurality of pVIII major coat proteins for the purpose of forming disulfide bonds. The present disclosure includes a method of engineering a Cys-Xaa-(Xaa)ₙ-Cys (CX(X)ₙC) (SEQ ID NO: 1) amino acid motif, where n = 1-8, on at least a plurality of peptides expressed at a surface of a genetically engineered bacteriophage, for the purpose of forming intra-peptide disulfide bonds initially, e.g., prior to crosslinking. The present disclosure can include a method of engineering a CX(X)ₙC (SEQ ID NO: 1) amino acid motif, where n = 1-8, on at least a plurality of pVIII major coat proteins of the bacteriophage. The genetically engineered bacteriophage as disclosed herein can further include a peptide that may or may not include at least two (e.g., 2, 3, 4, 5 or more) cysteine residues expressed on a plurality of external coat proteins, e.g., pIII, pIX, or pVIII, or a combination thereof (e.g., a cytokine, e.g., IL-2, on pVIII, an antibody fragment, e.g., anti-PD-1/PD-L1 fragment, on pIII, and/or a fluorescent protein, e.g., GFP, on pIX). The present disclosure provides methods for crosslinking the genetically engineered bacteriophage and compositions thereof to form solid materials, e.g., nanofoams or hydrogels, using exposure to light, a photoinitiator, a reduction agent, e.g., reducing agent, or a combination thereof. The present disclosure further provides methods to convert these phage-based solid materials, e.g., nanofoams or hydrogels, back into a liquid form. The present disclosure also provides methods for reversibly crosslinking the genetically engineered bacteriophage as disclosed herein and compositions thereof to form a solid material, e.g., nanofoam or hydrogel, in the presence of at least one analyte, e.g., pathogen or molecule of interest. The genetically engineered bacteriophage as disclosed herein, compositions thereof, and phage-based solid materials, e.g., nanofoams or hydrogels, can increase or decrease their fluorescence and/or absorbance in the presence of the at least one analyte, e.g., pathogen or molecule of interest. The present disclosure also relates to compositions comprising at least one genetically engineered bacteriophage.

### Definitions

Where values are described as ranges, endpoints are included. Furthermore, it will be understood that such disclosure includes the disclosure of all possible sub-ranges within such ranges, as well as specific numerical values that fall within such ranges irrespective of whether a specific numerical value or specific sub-range is expressly stated.

The terms “proteins” and “peptides” are used interchangeably and refer to a sequence or sequences of amino acids linked together via peptide bonds.

The term “about,” as used herein, refers to +/- 10% of a recited value.

The term “plurality,” as used herein, refers to more than one.

The term “substantially homogeneous,” as used herein, refers to +/- 5% variance. One of ordinary skill in the biological arts will understand that biological and chemical phenomena rarely, if ever, go to completion, and/or proceed to completeness, or achieve or avoid an absolute result. The term “substantially” is therefore used herein to capture the potential lack of completeness or totality inherent in many biological and chemical phenomena.

The term “genetically engineered,” as used herein, refers to pre-designed or intentional modifications to a primary nucleic acid or amino acid sequence that vary from a starting point, such as a wild-type sequence. Often, these modifications are designed to induce a phenotypic change, whether at a molecular, cellular, tissue, organ, and/or organismal level, that results in a change in feature or property, whether structural, chemical, and/or functional.

The term “operably linked,” as used herein, refers to a functional connection between two or more molecules, constructs, transcripts, entities, moieties, or genomic or genetic elements. For example, a gene of interest that is operably linked to an inducible promoter allows transcription of the gene of interest under conditions where the promoter is induced or activated.

The term “nanofoam,” as used herein, refers to nanostructured porous materials with a significant population of pores less than 100 nm in diameter.

The term “bacteriophage-templated nanofoam,” as used herein, refers to nanostructured porous materials made from crosslinked bacteriophage with a significant population of pores less than 100 nm in diameter.

The term “solid,” as used herein, refers to a material that has a stable shape, for example, a bacteriophage based hydrogel or nanofoam.

The term “intra-peptide disulfide bond,” as used herein, refers to a sulfur-sulfur bond between two cysteine amino acids located on the same peptide chain.

The term “inter-peptide disulfide bond,” as used herein, refers to a sulfur-sulfur bond between two cysteine amino acids located on two or more different peptide chains in different bacteriophage.

The terms “amino acid,” “residue,” or “amino acid residue” are used interchangeably and, as used herein, refer to an organic compound that contains a central carbon, an amino group, a carboxyl group and a side chain group specific to each amino acid.

The term “thiol group,” as used herein, refers to an organic sulfur compound containing a at least one carbon bonded to a sulfur atom that is either protonated (e.g., sulfhydryl group) or not protonated (e.g., thiolate). A thiol group can be found in the amino acid cysteine and other molecules. Two thiol groups can bond to form a disulfide bond.

The term “proximate,” as used herein, refers to a peptide sequence (e.g., a peptide of a plurality of peptides having two or more cysteine residues) close to the N- or C-terminus of a protein or moiety (e.g., a pVIII major coat protein). Proximal to the N- or C-terminus of the protein or moiety (e.g., a pVIII major coat protein) is adjacent (proximate) to the N- or C-terminus of the protein or moiety or the noncoding region originally associated therewith, or it means that it is in contact with or adjacent to the non-encrypted area originally associated with. Alternatively, proximal can mean close to the protein or moiety (e.g., a pVIII major coat protein), such that there is no coding sequence between the N- or C-terminus of the protein or moiety (e.g., a pVIII major coat protein) and the peptide sequence (e.g., a peptide of a plurality of peptides having two or more cysteine residues). For example, proximate can be within one, two, three, four, five, six, seven, eight, nine, ten, eleven, twelve, thirteen, fourteen, or fifteen amino acid residues.

The term “distal,” as used herein, refers to a peptide sequence far from the N- or C-terminus of a protein or moiety. Distal to the N- or C-terminus of the protein or moiety is far from (distal to) the N- or C-terminus of the protein or moiety or the noncoding region originally associated therewith, or it means that it is not in contact with or not adjacent to the non-encrypted area originally associated therewith. Alternatively, distal can mean far from the protein or moiety (e.g., a pVIII major coat protein), such that there is at least one coding sequence between the Nor C-terminus of the protein or moiety (e.g., a pVIII major coat protein) and the peptide sequence (e.g., a peptide of a plurality of peptides having two or more cysteine residues). For example, distal can be at least within 16 amino acid residues or more. In some embodiments, the secondary sequence (e.g., a protease-cleavable amino acid sequence) is upstream of the amino acid sequence comprising the plurality of peptides containing two or more cysteine residues. In some embodiments, the secondary sequence (e.g., a protease-cleavable amino acid sequence) is downstream of the amino acid sequence comprising the plurality of peptides containing two or more cysteine residues. In some embodiments, the secondary sequence is a single amino acid residue recognized by at least one protease. In some embodiments, the single amino acid residue can be upstream of the amino acid sequence comprising the plurality of peptides containing two or more cysteine residues. In some embodiments, the single amino acid residue can be downstream of the amino acid sequence comprising the plurality of peptides containing two or more cysteine residues. In some embodiments, the single amino acid residue is encompassed in the same amino acid sequence comprising the plurality of peptides containing two or more cysteine residues.

The terms “structurally substantially the same” and “structurally similar,” are used interchangeably and refer to nucleotide or amino acid sequences having a sequence identity of more than 50% compared to another sequence, for example to a pVIII surface protein of a bacteriophage. In some embodiments, a structurally similar amino acid sequence has at least 50% sequence identity, or at least 55%, or at least 60%, or at least 65%, or at least 70%, or at least 75%, or at least 80%, or at least 85%, or at least 90%, or 92%, or 95%, 97%, or 98%, or 99% sequence identity. In some embodiments, a structurally similar sequence, can be used to refer to a sequence that is homologous to another sequence. For example, a homologous sequence has at least 70%, or at least 75%, or at least 80%, or at least 85%, or at least 90%, or 92%, or 95%, 97%, or 98% sequence identity to another sequence. In some embodiments, a homologous sequence has a nucleotide sequence of about 15 to about 30 nucleotides that overlaps, i.e., is homologous, to the nucleotide sequence of another nucleotide sequence.

The term “structurally distinct” refers to an amino acid sequence that has a sequence identity of less than 50% compared to another sequence, for example to a pVIII surface protein of a bacteriophage. In some embodiments, a structurally distinct amino acid sequence has less than 50% sequence identity, or less than 45%, or less than 40%, or less than 35%, or less than 30%, or less than 25%, or less than 20% sequence identity.

The terms “substantially the same function” and “similar function,” are used interchangeably and refer to a peptide that, while it may or may not have structural similarity to a second peptide, the biological result is the same. For example, DNA polymerase I (Pol I) enzyme and Pol y (gamma) are prokaryotic and eukaryotic polymerases, respectively, and while their sequence or structure is different their ultimate biological function achieves a similar result: synthesis of DNA from deoxyribonucleotides. In some embodiments, a protein with a similar function to a second protein has a structurally similar primary sequence to said second protein. In some embodiments, a protein with a similar function to a second protein has a structurally distinct primary sequence to said second protein.

The terms “functionally distinct” and “distinct function,” are used interchangeably and, as used herein, refer to a peptide that, while it may or may not have structural similarity to a second peptide, the biological result is different. In some embodiments, a protein that is functionally distinct to a second protein has a structurally similar primary sequence compared to said second protein. In some embodiments, a protein that is functionally distinct to a second protein has a structurally distinct primary sequence to said second protein.

The term “crosslinking,” as used herein, refers to one or more bonding interactions between two or more moieties, either directly or via one or more additional moieties that serves as a linking agent, to form a structure that is sufficiently stable so that the moieties remain physically associated or connected under the conditions in which the structure is used, e.g., physiological conditions or conditions of high temperature and high pressure. The bonding interactions may be covalent bonding, ionic bonding, hydrogen bonding, hybridization-based connectivity, hydrophobic interactions or combinations thereof.

The term “crosslinked gel,” as used herein, refers to a hydrogel structure formed by crosslinking moieties that remain physically associated or connected under the conditions in which the structure is used, e.g., physiological conditions or conditions of high temperature and high pressure. The bonding interactions may be covalent bonding, ionic bonding, hydrogen bonding, hybridization-based connectivity, hydrophobic interactions or combinations thereof.

A “reduction agent” can be an agent that participates in the sequence of events leading to crosslinking the phage by donating electrons, but does not participate in the final inter-peptide bond formed because it is consumed.

A “disulfide crosslinking agent” can be an agent that serves as a physical crosslink between cysteine residues by participating in the covalent bond formation between cysteines of different phage (inter-peptide disulfides).

The terms “Cys-Xaa-(Xaa)ₙ-Cys (SEQ ID NO: 1) motif” or “CX(X)ₙC (SEQ ID NO: 1) motif,” where n = 1, 2, 3, 4, 5, 6, 7, or 8, are used interchangeably and refer to a series of four or more bonded amino acids in which the first and last amino acids are cysteine residues and each of the second, third, and up to n^{th} amino acids, independently, can be any amino acid from the 20 natural amino acids, selenocysteine, pyrrolysine and/or a synthetic amino acid.

A “distinct moiety,” as used herein, refers to a moiety that has a specific sequence, use and/or function and said sequence, use, and/or function is different from the peptides described herein. In some embodiments, the distinct moiety is a peptide that is different from the peptides comprising two or more cysteine residues and/or a pVIII protein. In some embodiments, the distinct moiety is a non-peptide moiety, for example an inorganic molecules or nanoparticles.

The term “therapeutic agent,” as used herein, refers to an agent, such as a formulation of the genetically engineered bacteriophage as disclosed herein or composition thereof, that can be used in the treatment, management, or control of one or more diseases or disorders (e.g., co-morbidities).

The term “treatment,” as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and improved prognosis. In some embodiments, formulations of the genetically engineered bacteriophages are used to treat cancer (e.g., ovarian cancer, breast cancer, pancreatic cancer, glioblastoma) or chronic inflammatory conditions (e.g., autoimmune diseases, e.g., autoinflammatory diseases, e.g., rheumatoid arthritis).

The term “therapeutically effective amount,” as used herein, refers to any amount or dosage of a therapeutic agent, such as a formulation of the genetically engineered bacteriophage as disclosed herein or composition(s) thereof, that is administered for periods of time necessary that is sufficient to achieve elimination, amelioration, slowing, lessening, or decrease in the severity of the symptoms associated with and/or the underlying cause of a disease or disorder in a subject receiving the agent. Formulations and compositions of the genetically engineered bacteriophage as disclosed herein are administered by any suitable means, including oral, intravenous, subcutaneous, intraperitoneal, intramuscular, intraarterial, intrapulmonary, intrathecal, intranasal, topical, systemic, local, intralesional, and/or intratumoral administration.

The terms “subject” or “patient,” as used herein, refer to any organism, e.g., animal, plant, or microbe, to which compositions as disclosed herein may be administered, e.g., for experimental, diagnostic, prophylactic, and/or therapeutic purposes. A subject or patient may also refer to any mammal, e.g., a human, who is being treated for a disease, e.g., cancer or inflammatory condition, and may be one who has been diagnosed by a medical or veterinary clinician as the case may be as having such a condition. Diagnosis may be performed by any suitable means. Patients as disclosed herein may have been subjected to standard tests or may have been identified, without examination, as one at high risk of having or developing a disease, e.g., cancer or inflammatory condition, due to the presence of one or more risk factors, such as age, genetics, or family history.

The term “isotopically labeled form,” as used herein refers to the inclusion of an isotopic form of one or more atoms in a molecule that is different from the naturally occurring isotopic distribution of the one or more atoms in nature. All isotopic forms are included as options in the composition of any molecule, protein, or peptide, unless a specific isotopic form of an atom is indicated.

In the event of any term having an inconsistent definition between this application and a referenced document, the term is to be interpreted as defined herein.

### Crosslinking

Exposed cysteine residues on a surface of a bacteriophage can be arranged to provide crosslinking groups. The thiol groups can be arranged on the surface to form disulfide bonds with other thiol groups on the surface of the bacteriophage. In particular, the thiol groups are arranged on each major coat protein on the surface to initially form disulfide bonds with the other thiol groups on the same coat protein, forming intra-peptide disulfide bonds. By forming these intra-peptide disulfide bonds within the same protein, a pVIII coat protein does not bind with another pVIII coat protein at the time a bacteriophage is formed. The intra-peptide disulfide bonds can be converted to inter-peptide disulfide bonds when bacteriophages and/or crosslinking agents are brought in proximity to each other, for example, during the formation of a solid material, e.g., nanofoam or hydrogel. Crosslinking through formation of inter-peptide disulfide bonds can occur by exposure to light, a photoinitiator, or a reduction agent (or a combination thereof). This crosslinking step can be controlled to allow a variety of solid materials, e.g., nanofoams or hydrogels, to be formed. Crosslinking can involve a crosslinking agent such as norbornene, maleimide, another alkene (for example, a substituted or unsubstituted carbon-carbon double bond moiety), an acrylate or a functional derivative thereof. In certain circumstances, the alkene can be an allylamine, an allyl alcohol, or an allyl acetate. In certain circumstances, the acrylate can be acrylamide, acrylic acid, PEGDA (poly(ethylene glycol diacrylate)), pentaerythritol triacrylate, glycerol propoxylate triacrylate, zirconium bromonorbornanelactone carboxylate triacrylate (PRM30), or Tris(2-hydroxyethyl) isocyanurate triacrylate.

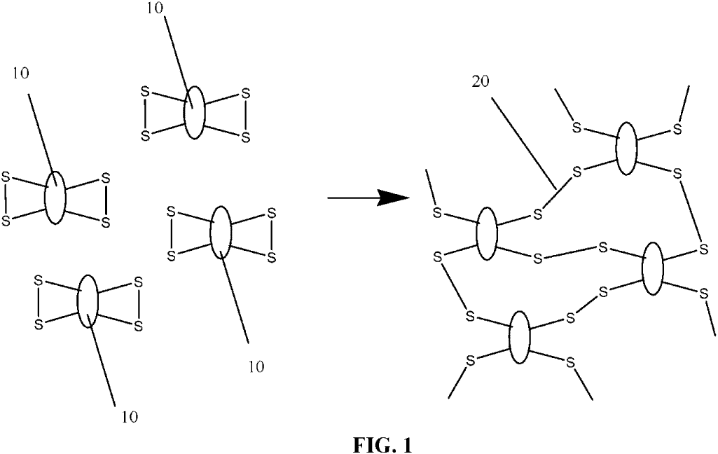

An example of an embodiment of a method as disclosed herein is shown in FIG. 1, where a method of forming a solid material 20, e.g., nanofoam or hydrogel, includes crosslinking a plurality of genetically engineered bacteriophages 10. For example, two or more cysteine residues form intra-peptide disulfide bonds within each major coat protein prior to crosslinking as exemplified in genetically engineered bacteriophages 10. The two or more cysteine residues then form inter-peptide disulfide bonds between major coat proteins of at least two different bacteriophages after crosslinking to form solid material 20, e.g., nanofoam or hydrogel. Each genetically engineered bacteriophage can include major coat proteins each having two or more cysteine residues expressed at a surface of the bacteriophage. By reduction, irradiation with light, and/or free radical initiation, crosslinks form to create the solid material, for example, a nanofoam or hydrogel. Once an intra-peptide disulfide bond is reduced, the thiols can reshuffle and form new disulfide bonds with thiols on adjacent phage coat proteins, forming inter-peptide disulfides, and a crosslinked network of bacteriophage or a phage-based solid material, e.g., nanofoam or hydrogel, is formed. The time it takes to form such a solid material, e.g., nanofoam or hydrogel, can range from seconds to minutes to hours. The solidification or gelation time can be adjusted (e.g., increased or decreased) by the amount of reduction agent added and/or the type of reduction agent used. This adjustment can allow temporal control of solidification, e.g., gelation, e.g., gelling, according to a required application. In certain embodiments, solidification, e.g., gelation, e.g., gelling, by a particular reduction agent can allow for the solid, e.g., nanofoam, e.g., hydrogel, e.g., gel, formation to be reversible. Addition of a higher concentration of reduction agent can break the inter-peptide disulfide crosslinking and return the solid material, e.g., nanofoam or hydrogel, to a liquid form.

The solid material formation method of the present disclosure can be used in bulk synthesis of material or controlled synthesis, such as ink jet printing or 3D printing.

The light irradiation can be ultraviolet, visible, or infrared light (e.g., near-infrared I, II or III).

The photoinitiator can be a phosphinate (such as lithium phenyl(2,4,6-trimethylbenzoyl)phosphinate (TPO)), a fluorescent dye (such as Eosin Y, or methylene blue), Irgacure 10 2959 (a propiophenone), or bacteriochlorophyll derivative.

The reduction agent can be a phosphine (such as tris(2-carboxyethyl)phosphine or tris(hydroxypropyl)phosphine) or a thiol (such as dithiothreitol). The activity of the reduction agent can be enhanced in the presence of a metal catalyst, such as copper).

In certain circumstances, a genetically engineered bacteriophage can include a plurality of proteins having two or more cysteine residues expressed at a surface of the bacteriophage. The two or more cysteine residues can form intra-peptide disulfides within each protein. In particular, the plurality of proteins can include a pVIII major coat protein.

The pVIII major coat protein can include two or more cysteine residues that form an intra-peptide disulfide bond. For example, the pVIII major coat protein can include a CX(X)ₙC (SEQ ID NO: 1) motif, where n = 1-8, proximate to the N-terminus of the protein, or -cysteine-amino acid-amino acid-cysteine (where each X is another amino acid residue). This motif includes two cysteine residues separated by two amino acid residues.

In some embodiments, the CX(X)ₙC (SEQ ID NO: 1) motif can be a CX(X)ₙC (SEQ ID NO: 1) motif, where n is 1, 2, 3, 4, 5, 6, 7, or 8. In some embodiments, the CX(X)ₙC (SEQ ID NO: 1) motif can be a 5, 6, 7, 8, 9, 10, 11 or 12 amino acid sequence that can be fused to the plurality of proteins expressed at the surface of the bacteriophage. In some embodiments, the cysteine (Cys or C) residues can be replaced with selenocysteine (Sec or U) residues. In some embodiments, the CX(X)ₙC has an n = 3, 4, 5, or 6. Each amino acid residue (Xaa or X) can be alanine (Ala or A), arginine (Arg or R), asparagine (Asn or N), aspartic acid (Asp or D), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), pyrrolysine, selenocysteine, serine (Ser or S), threonine (Thr or T), tryptophan (Trp or W), tyrosine (Tyr or Y), valine (Val or V), or a synthetic amino acid. A synthetic amino acid can be an amino acid that is not naturally occurring. In some embodiments, the amino acids of peptides expressed as a fusion to the pVIII protein can be constructed to include two fixed cysteine residues separated by two randomized amino acid residues, while the other amino acids in the peptide are randomized. A sequence that conveys the right reduction potential for intra-peptide disulfides to form, rather than inter-subunit disulfides (e.g., disulfides between cysteine residues on neighboring polypeptide chains on the same bacteriophage), allows viral phage particles to form. The conserved sequence(s) of the pVIII fusion peptide required for phage assembly with a high concentration of cysteine residues can be identified by methods known in the art, such as DNA sequencing. These approaches enable expression of at least two cysteine residues on all pVIII proteins.

In some embodiments, the CX(X)ₙC (SEQ ID NO: 1) motif, where n = 1, is included in a sequence of Xaa-Cys-Pro-Asp-Cys-Xaa-Xaa-Xaa (XCPDCXXX) (SEQ ID NO: 2), wherein each Xaa or X is an amino acid residue, Cys or C is cysteine, Pro or P is proline, and Asp or D is aspartic acid. In some embodiments, two cysteine residues can be separated by proline and aspartic acid.

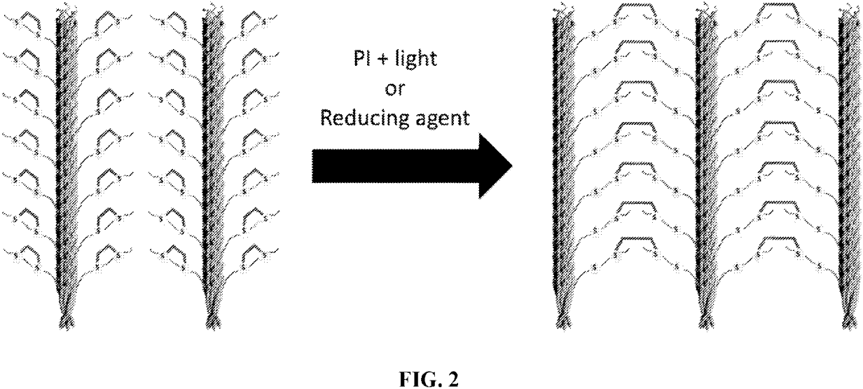

FIG. 2 shows a schematic of cysteine-based phage crosslinking. M13 bacteriophage expressing peptides with a Cys-Pro-Asp-Cys (CPDC) (SEQ ID NO: 3) sequence on each pVIII major coat protein. The cysteine residues (yellow) in the Cys-Pro-Asp-Cys (CPDC) (SEQ ID NO: 3) sequence form an intra-peptide disulfide bond within each pVIII. When a photoinitiator (PI) is added and light is shined at the right wavelength for the particular PI, or when a reducing agent strong enough to open the intra-peptide disulfides is added to the solution then disulfides are reshuffled and form inter-peptide disulfides between pVIII of neighboring phage creating a crosslinked network of phage (a phage hydrogel). Using a light-based crosslinking technique may also form other bonds in addition to disulfides such as carbon sulfur bonds or sulfur oxygen bonds.

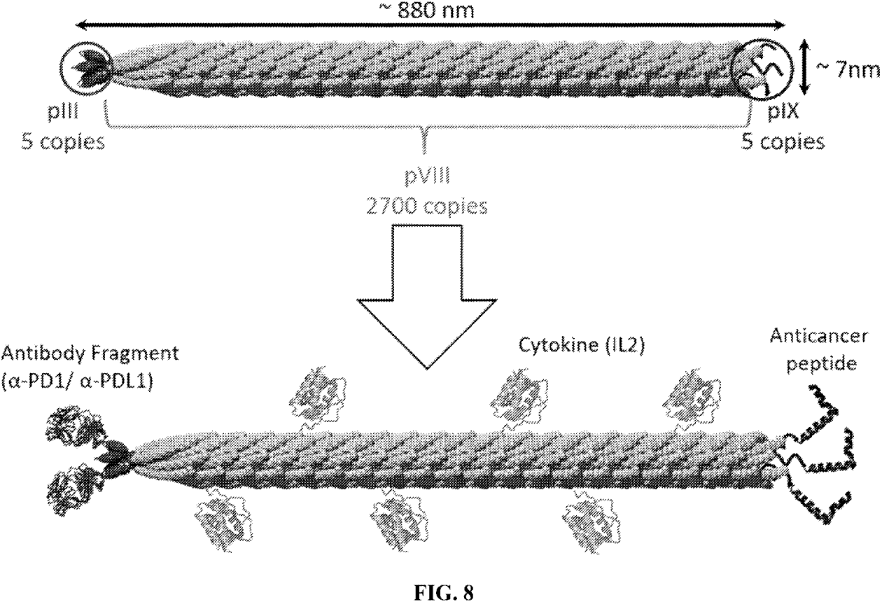

Additional peptides can be incorporated in conjunction with the plurality of pVIII proteins expressing a CX(X)ₙC motif (SEQ ID NO: 1), where n = 1-8. An additional pVIII gene can be added to the bacteriophage genome already containing a pVIII gene coding for a pVIII fused to CX(X)ₙC (SEQ ID NO: 1) motif, where n = 1-8. Such a genetic construct can allow for a bacteriophage that harbors two or more different populations of pVIII fusion proteins. This particular construct would, for example, have (a) one pVIII fusion protein population that is fused to a distinct moiety. In some embodiments, the distinct moiety is a peptide designed for a specific purpose, such as a therapeutic purpose, or to a scaffold or preferential reactive site for materials synthesis. The (b) other pVIII fusion protein population would have a CX(X)ₙC (SEQ ID NO: 1) motif, where n = 1-8, that allows crosslinking of the genetically engineered bacteriophage into a hydrogel and/or attaching molecules onto the thiols associated with the bacteriophage. FIG. 8 is an exemplary schematic of a bacteriophage containing different therapeutic proteins that can be expressed as fusions to three different bacteriophage coat proteins. In some embodiments, the (a) pVIII fusion protein that is fused to a peptide is a structurally different or has a different sequence compared to the (b) pVIII fusion protein population that has the CX(X)ₙC (SEQ ID NO: 1) motif, where n is an integer from 1 to 8. In some embodiments, the distinct moieties, such as a peptide designed for a specific purpose, such as a therapeutic purpose, or to a scaffold or preferential reactive site for materials synthesis can be a targeting moiety, an antibody, an antibody fragment, a bi-specific T-cell engager, an affibody, a nanobody, a cell penetrating peptide, a cytokine, a growth factor, a DNA repair enzyme, an opioid receptor-binding peptide, a protease, a hormone, a biotinylation protein, an antibiotic resistance gene, a bioluminescent protein, a fluorescent protein, and/or a chemiluminescent protein. In some embodiments, the antibody can be anti-PD-1, anti-PD-L1, or anti-CTLA4. In some embodiments, the antibody fragment can be a single-chain variable antibody fragment. In some embodiments, the cytokine can be IL-2, IL-7, IL-18, or IL-27. In some embodiments, the growth factor can be IGF, NGF, GDNF, FGF, VEGF, TGF-alpha fragment, TGF-beta fragment, PDGF, or macrophage activator. In some embodiments, the DNA repair enzyme can be endonuclease V. In some embodiments, the opioid receptor-binding peptide can be enkephalin or substance P. In some embodiments, the hormone can insulin, glucagon, ghrelin, angiotensin, or thyroid-stimulating hormone (TSH). In some embodiments, the biotinylation protein can be BirA. In some embodiments, the bioluminescent protein can be aequorin, firefly luciferase, Renilla luciferase, red luciferase, or nanoluciferase. In some embodiments, the fluorescent protein can be EGFP, EYFP, ECFP, superfolder GFP, dsRed, mCherry, mOrange, mOrange2, mRaspberry, mTangerine, mApple, mRuby, mPlum, mKate1, mKate2, mKO2, mNeptune, mNeptune681, mNeptune684, mTurquoise, TagBFP, TagRFP675, azurite, EBFP2, mKalama1, iRFP682, iRFP713, iRFP720, miRFP703, miRFP670, miRFP670nano, miRFP682, miRFP702, miRFP703, miRFP709, miRFP713, miRFP720, iBlueberry, Wi-Phy, or mIFP. In some embodiments, the chemiluminescent protein can be β galactosidase, alkaline phosphatase, or horseradish peroxidase (HRP).

A construct as immediately described above, allows for additional functionalities to be added to the bacteriophage, these functionalities derived from the distinct moiety, such as therapeutic and/or targeting peptides/proteins, peptides/proteins that can be used for material deposition and synthesis, or small molecule moieties. Once these hybrid pVIII bacteriophage are produced, the CX(X)ₙC (SEQ ID NO: 1) motif, where n = 1-8, that is expressed can be used for crosslinking and making novel types of hydrogels presenting different types of peptides or proteins. These hydrogels can be used in therapeutic applications or for materials synthesis. The additional pVIII fusion genetic construct can be added to the bacteriophage genome or expressed on a separate phagemid plasmid. The additional population of pVIII fusion proteins can be expressed under Lac or Tac promoters and induced by IPTG, or can be expressed under constitutive promotors that would generate the protein in a continuous manner once the host bacteria are infected with the genetically engineered bacteriophage.

The genetically engineered bacteriophage as disclosed herein can be used to make a conjugate. The conjugate can be a plurality of a second molecule linked to a surface of the bacteriophage via a disulfide bond, where the disulfide bond is made by cleavage of the intra-peptide disulfide bond. The second molecule can be a di-thiol compound or a maleimide compound. The conjugate can be a polymer of the bacteriophage and the second molecule. In certain circumstances, the method can further include adding a disulfide crosslinking agent to crosslink the plurality of genetically engineered bacteriophages.

In another aspect, a solid material can include the genetically engineered bacteriophage crosslinked to other genetically engineered bacteriophages and/or a di-thiol compound. The solid material can be a hydrogel or a hybrid material templated from the genetically engineered bacteriophage. For example, the solid material can be an inorganic material, such as a perovskite, hydroxyapatite, a transition metal oxide, a transition metal carbide, or a transition metal nitride, such as vanadium nitride. For example, the solid material can include a disulfide crosslinking agent crosslinking the plurality of genetically engineered bacteriophage. The disulfide crosslinking agent can be an organic molecule with at least two thiol moieties capable of forming disulfide bonds.

Bacteriophage expressing a set of proteins, such as pVIII, pIII and/or pIX, can be mixed with one or more bacteriophage expressing a different set of proteins fused to the same coat proteins. Mixing and matching bacteriophage having different sets of surface proteins allows for malleability of the hydrogel to a specific requirement, for example as a therapeutic for a specific disease or disease state or for synthesizing materials that vary in composition.

Chemical conjugation of molecules to the bacteriophage surface is also possible. Conjugation of molecules such as fluorophores, catalysts, polymers, polysaccharides, DNA, positively charged molecules (such as methyl ammonium containing molecules) and amphiphilic molecules can be done through covalent attachment with the thiols of the CX(X)ₙC (SEQ ID NO: 1) motif, where n = 1-8, (e.g., maleimide reactions), through coordinative bonds to the thiols or through functionalization of primary amines, carboxylic acids or hydroxyl groups present on the bacteriophage (e.g., EDC chemistry). Two or more molecules can be conjugated at the same time with orthogonal chemistries (e.g., maleimide and EDC). Chemically conjugating molecules onto the bacteriophage allows for hydrogels formed from the modified bacteriophage to have certain characteristics. Conjugating polymers or surfactants onto the bacteriophage can alter the properties of the hydrogel, for example by making it more flexible or more robust. Conjugating catalysts onto the bacteriophage may provide the hydrogel’s ability to catalyze reactions for synthesis of small molecules (e.g., hydrogen, alcohols etc.) and even polymers. Anti-microbial glycans can be conjugated onto the bacteriophage to produce anti-microbial hydrogels. Conjugating small molecules onto the bacteriophage, such as TLR7/8a, that can elicit immune responses through Toll-Like Receptor agonists (TLR), may result in hydrogels that can boost the immune response against tumors.

Materials can also be infused into the gel for different applications. For example drugs in liquid or solid form can be mixed into the gel for therapeutic purposes that would complement or enhance the therapeutic activity of drugs and/or peptides expressed on the body of the bacteriophage. Gold nanoparticles, carbon nanotubes or magnetic nanoparticles can be added to actively degrade the hydrogel or to allow for degradation of the hydrogel via some other means. Release of drugs, or other cargos, from the hydrogel can be achieved by using either light (visible or infrared) or a magnetic field.

The bacteriophage hydrogel can also be used in the detection of pathogens in a subject or entity. The bacteriophage pVIII coat protein can be engineered to express peptides that bind specific pathogens with high affinity. A library of bacteriophage can also be panned or screened to find an optimal binding molecule for a specific pathogen. The pathogen can be captured and concentrated from blood or bodily fluids containing said pathogen. The hydrogel can then be reversibly turned into liquid form, which can be further analyzed for the pathogen of interest. Molecules or nanoparticles that can sense and signal (through fluorescence or absorbance) the presence of a molecule or pathogen can also be incorporated into the hydrogel for in situ determination of the existence of said molecule or pathogen in the tested sample. In some embodiments, the pathogens can be bacteria, fungi, microalgae, yeast, viruses, protozoa, parasites, and/or insects. In some embodiments, the subject or entity can be humans, animals, plants, rodents, mammals, non-human primates, pigs, horses, and/or dogs.

The bacteriophage hydrogel can also be used in the detection of analytes.

Detecting can be accomplished, for example, when the solid material and/or the moiety capable of binding and/or recognizing the entity can have a change in the fluorescence or absorbance (e.g., fluorophore, quantum dot, nanoparticle, rare-earth up/down-conversion nanoparticles, single-walled carbon nanotubes (SWCNTs)) when the analyte is present in an added component. For example, it is possible for hydrogel formation both before and after contacting the analyte.

In another example, detecting can be accomplished, when the recognition and/or binding moieties are genetically engineered on the phage, in which case they can be chemically functionalized onto phage (e.g., polysaccharides) or they can be two different moieties with separate recognition and binding functions.

In another example, detecting can be accomplished when the solid material can undergo a phase change to a liquid, fluid, and/or gas upon contacting the analyte.

In another example, detecting can be accomplished when the solid material and/or the moiety capable of binding and/or recognizing the analyte can undergo a change in surface plasmon resonance, can become surface plasmon resonance active, or can change temperature (e.g., measured via isothermal titration calorimetry) or pH.

The bacteriophage pVIII coat protein can be engineered to express peptides that bind specific analytes with high affinity. A library of bacteriophage can also be panned or screened to find an optimal binding molecule for a specific analyte. The hydrogel can then be reversibly turned into liquid form, which can be further analyzed for the analyte of interest. Molecules or nanoparticles that can sense and signal (through fluorescence or absorbance) the presence of an analyte can also be incorporated into the hydrogel for in situ determination of the existence of said analyte in the tested sample. In some embodiments, the analytes can include toxic industrial chemicals, chemical warfare agents, cancer cells, antibiotics, ions, small molecules, biomolecules (e.g., uric acid, hydrogen peroxide, glucose), proteins, nucleic acids, enzymes, peptides, RNA, polysaccharides, glycans, glycoproteins, cell metabolites, siderophores, aflatoxins, metal ions, crown ethers, change in temperature, pH, or electric or magnetic fields, and/or light. In some embodiments, the analytes can be in a fluid such as ocular, oral, nasal, ocular, genital, rectal, glandular, lymph, lesional, or blister fluid, or blood, plasma, or serum.

The bacteriophage hydrogel can also shrink or swell in size, change their crosslinking density, change optical (e.g., color, opacity, diffraction wavelength), thermal, electrochemical, electrical, and/or mechanical properties, and/or release embedded nanoparticles or moieties in response to an analyte. These effects may be reversible, as in shape-memory hydrogels.

The genetically engineered bacteriophage as disclosed herein can be made by expressing a plurality of proteins having two or more cysteine residues at a surface of the bacteriophage. As described above, the proteins can include a CX(X)ₙC (SEQ ID NO: 1) motif, where n = 1-8, at the surface of the bacteriophage.

In certain circumstances, a reduction agent can be added to crosslink the plurality of genetically engineered bacteriophages.

One of the features described herein is that a solid material can be made by crosslinking, including disulfide reshuffling from an intra-peptide disulfide to an inter-peptide disulfide. In some examples, crosslinking can include reduction followed by disulfide reshuffling.

Herein, a genetically engineered M13 bacteriophage is described with a high effective concentration of cysteine residues expressed on the pVIII major coat protein. Expressing a high degree of cysteine residues on the surface of the M13 bacteriophage has not been feasible before despite attempts by many researchers. These cysteine residues can be used as handles to conjugate different molecules and can also be used to crosslink between bacteriophage to make genetically engineerable hydrogels.

Expressing cysteine residues on all pVIII proteins of the bacteriophage is thought to prevent virus formation. See, for example, Zwick MB, Shen J, Scott JK. Homodimeric peptides displayed by the major coat protein of filamentous phage. Journal of molecular biology 2000, 300(2): 307-320, which is incorporated by reference in its entirety. The pVIII protein is expressed in the E. coli cytoplasm and inserted into the cytoplasmic membrane with the N-terminal 20 amino acids exposed in the periplasm. The disulfide forming machinery residing in the periplasm can thus form disulfide bonds between cysteine residues present on neighboring pVIII proteins. A working hypothesis was that a large number of inter-subunit disulfide connections between pVIII proteins could restrict their ability to assemble in the correct spatial arrangement relative to one another, thus preventing the formation of a mature bacteriophage particle. In order to prevent these putative inter-subunit disulfides from forming between neighboring cysteine-containing pVIII proteins on the same bacteriophage, a pVIII fusion peptide was genetically modified to express a CX(X)ₙC (SEQ ID NO: 1) motif, where n = 1-8, which could preferentially form intra-peptide disulfides within each peptide rather than inter-peptide disulfides between adjacent pVIII subunits. The reduction potential of the CX(X)ₙC (SEQ ID NO: 1) motifs, where n = 1-8, has been studied thoroughly in proteins with thioredoxin-like folds. See, for example, Krause G, Lundstrom J, Barea JL, Pueyo de la Cuesta C, Holmgren A. Mimicking the active site of protein disulfide-isomerase by substitution of proline 34 in Escherichia coli thioredoxin. The Journal of biological chemistry 1991, 266(15): 9494-9500; Chivers PT, Prehoda KE, Raines RT. The CXXC motif: a rheostat in the active site. Biochemistry 1997, 36(14): 4061-4066; and Mossner E, Huber-Wunderlich M, Glockshuber R. Characterization of Escherichia coli thioredoxin variants mimicking the active-sites of other thiol/disulfide oxidoreductases. Protein science: a publication of the Protein Society 1998, 7(5): 1233-1244; each of which is incorporated by reference in its entirety. The two varying amino acids between the two cysteine residues significantly affect the reduction potential of the di-thiol, and hence its tendency to form a disulfide.

To utilize this phenomenon to make an embodiment of the genetically engineered bacteriophage as disclosed herein with cysteine residues expressed on the pVIII coat protein, libraries of genetically engineered bacteriophages whose genomes encode eight amino acid peptides expressed as a fusion to the pVIII protein were constructed. These peptides contain two fixed cysteine residues separated by two randomized amino acid residues, while the other amino acids in the peptide sequence were randomized. This sequence design was created with the rationale that any sequence that would possess the correct reduction potential for intra-peptide disulfide bonds to form, rather than inter-subunit disulfides, would allow mature viral phage particles to form. From these libraries, it was possible to identify sequences that enabled expression of cysteine residues on all pVIII proteins and still form viable bacteriophage particles. The conserved sequence of an eight amino acid peptide expressed as a fusion to the pVIII protein that was required for assembly of genetically engineered bacteriophages as disclosed herein with a high concentration of cysteine residues was XCPDCXXX (SEQ ID NO: 2).

Measurements of thiol content using 5,5′-dithiobis (2-nitrobenzoic acid)(DTNB) before, and after reduction and removal of the reduction agent, indicated that the cysteine residues were indeed disulfide bonded in intra-peptide disulfide bonds.

Cysteine residues located in peptides at or expressed on the virus surface can be used to specifically bind molecules by chemical conjugation. Since the cysteine positions are set, the position of the conjugated molecules on the virus surface can be pre-determined and it conforms to the viral helical scaffold. Molecules such as light harvesting molecules, fluorescent probes, catalysts, drugs, polymers, DNA, peptides and even enzymes can be conjugated in precise positions on the virus surface. In addition, amine groups on the pVIII protein can be used for orthogonal conjugation with other molecules placed at a precise distance from the cysteine-bound molecules. These two precisely positioned populations of molecules can be designed to ‘cross-talk,’ and can work in a synergistic fashion or in tandem for catalyst cascade reactions.

The cysteine residues can undergo chemical reaction to form inter-peptide disulfide bonds with cysteine residues of adjacent bacteriophage particles. For example, another type of possible cysteine chemistry is crosslinking between cysteine residues (e.g., thiols) of adjacent bacteriophage particles to create a hydrogel.

Two types of mechanisms can be used to crosslink the bacteriophage: 1) light-/photoinitiator-based crosslinking, and 2) reduction followed by disulfide reshuffling. In light-based crosslinking a photoinitiator is mixed in with the bacteriophage solution. When the right wavelength of light to be absorbed by the photoinitiator is shined on the solution the photoinitiator forms free radicals. These in turn form free radicals on a cysteine’s sulfur atom which can subsequently form a bond with another sulfur free radical (or other free radicals) on adjacent phages thus forming a network of crosslinked viruses. By using photoinitiators that can absorb at different wavelengths it is possible to tune the wavelength of light required for forming the hydrogel; from ultraviolet (UV) through visible to infrared (IR). Tuning the wavelength that is responsible for gelling can be useful in meeting the requirements of different applications. For example UV light or visible light can be used in 3D printing, while IR can be used if gelling is required inside the body in situ without the need for incision because of the deeper penetration of IR light through the body.

In addition to crosslinking directly between bacteriophage particles, norbornene (or other crosslinking moiety) bearing polymers can be used as crosslinkers as well. Norbornene can form a free radical when exposed to free radicals from a photoinitiator and subsequently form thiol-norbornene bonds. See, for example, Fairbanks BD, Schwartz MP, Halevi AE, Nuttelman CR, Bowman CN and Anseth KS, Advanced Materials, A Versatile Synthetic Extracellular Matrix Mimic via Thiol-Norbornene Photopolymerization 2009, 21, 5005-5010, which is incorporated by reference in its entirety. See also norbornene-based hydrogels described in Raza, Macromolecular Bioscience, 2013, 13, 1048-1058, which is incorporated by reference in its entirety.

In the second crosslinking mechanism a reduction agent strong enough to reduce the intra-peptide disulfides, such as tris(2-carboxyethyl)phosphine (TCEP), is added to the phage solution. Once the intra-peptide disulfide is reduced the disulfides can reshuffle and form disulfides with thiols on adjacent phage resulting in a crosslinked network of phage or a phage hydrogel. The time it takes to gel can be tuned from seconds to minutes to hours by changing the amount of reduction agent and/or the type of reduction agent. This tunability can allow temporal control of gelling according to the required application. In addition this chemical process is accelerated catalytically by copper which allows for another layer of control over gelling and may also allow spatial and temporal control of gelling in certain applications. In some embodiments, the method of forming a solid material can be catalyzed using copper metal. In some embodiments, the copper needle is compatible with three-dimensional (3D) printing. In some embodiments, the method of forming a solid material can be catalyzed in situ during local delivery or administration using a copper needle. In some embodiments, the copper needle is compatible with an endoscopic device for in situ local delivery or administration within the body. In some embodiments, the solid material is a hydrogel. In some embodiments, the copper needle is compatible with three-dimensional (3D) printing. In some embodiments, the copper needle is compatible with three-dimensional (3D) printing in situ, e.g., inside the body. In some embodiments, the genetic sequence of the genetically engineered bacteriophage as disclosed herein may allow for spatial and temporal control over in situ formation of a solid material during injection by the formation of disulfide bonds between cysteine residues and/or carbon-sulfur bonds. Exemplary needle gauges for the copper needle include 34, 33, 32, 31, 30, 29, 28, 27, 26s, 26, 25s, 25, 24, 23s, 23, 22s, 22, 21, 20, 19, 18, 17, 16, 15, 14, 13, 12, 11, or 10. Exemplary needle lengths (in inches ‘ ” ’) for the copper needle include 5/32″, 3/16″, 5/16″, 15/64″, ⅜″, ⅝″, ⅞″, ¾″, ½″, 1″, 1.25″, 1.5″, 2″, 2.5″, 6″, 7″, 8″, or 12″.

The ability to control hydrogel formation spatially and temporally permits the use of the hydrogel as a matrix for 3D printing structures. A stereolithography (SLA) or digital light processing (DLP) type 3D printer can be used for 3D printing of the genetically engineered bacteriophage hydrogel as disclosed herein using white light or a particular wavelength of light adjusted to the maximum absorbance of the photoinitiator used in some of the compositions as disclosed herein. A modified fused deposition (FDM) type 3D printer in which the heated nozzle is replaced with a copper needle can also be used for 3D printing with the genetically engineered bacteriophage as disclosed herein or compositions thereof using a reducing agent in the formulations to 3D print a hydrogel.

A device that can be inserted into the body that is fitted with a miniature xyz stage to move a copper needle or a needle adjusted with a light source can be used to 3D print tissue and organ scaffolds inside the body. The hydrogel used for 3D printing in the body can be genetically engineered and customized to the growth of a required tissue by genetically fusing the proteins of interest, e.g., growth factors, onto the plurality of proteins at a bacteriophage surface, thereby promoting differentiation and proliferation of cells into the desired tissue.

The bacteriophage based hydrogel as disclosed herein, being made of natural materials, i.e., protein and DNA, can be used in a variety of applications. In addition, a major advantage of these hydrogels is that they can be genetically engineered to present peptide or protein moieties tailored for the required application. These applications can be divided into two categories: 1) biomaterials in which the bacteriophage based hydrogel is used to interact with biological systems or has a biological significance, and 2) inorganic materials in which the bacteriophage based hydrogel is used as a template for synthesizing inorganic materials.

A number of biomaterial applications of the bacteriophage based hydrogel as disclosed herein are possible.

In one non-limiting example, a gel based drug delivery system can be loaded with many different kinds of drugs, molecules and biological elements (small molecule drugs, liposomes, drug powder, polymers, cytokines, CAR-T cells, antibiotics). These hydrogels, as described herein, can be delivered in situ while containing the desired drug or molecule. For example, a bacteriophage-based hydrogel containing chemotherapeutic drugs can be delivered directly at a tumor site, via intratumoral injection using a copper needle, negating the necessity for systematic drug delivery and thus potentially reducing unwanted side-effects. Adding the bacteriophage-based hydrogel/drug mixture can also be done after removal of a tumor mass in order to kill any leftover cancerous cells (e.g., residual tumor burden) not removed by surgery. In other instances, CAR-T cells within the hydrogel can be delivered to a tumor site, enabling the CAR-T cells to be delivered directly to a site of interest and in a way that promotes cell viability because of the hydrogel matrix environment. Drugs can be delivered in different formulations such as a free drug solution, inside liposomes or even as drug crystals/powder. In addition, this drug delivery system can be used for slow release of antibiotics or anti-inflammatory drugs for wound healing.

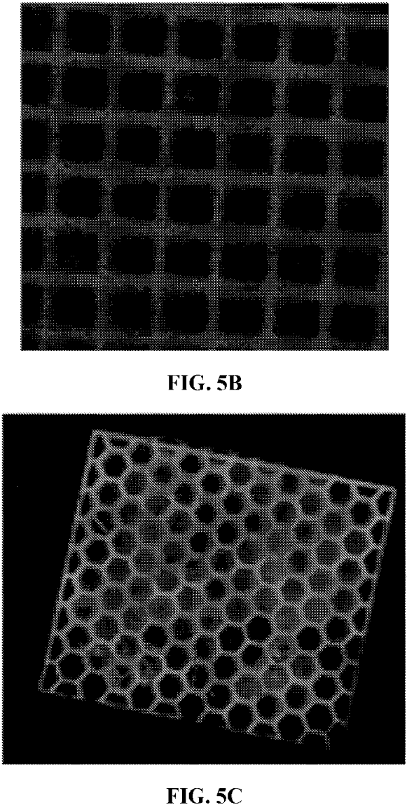

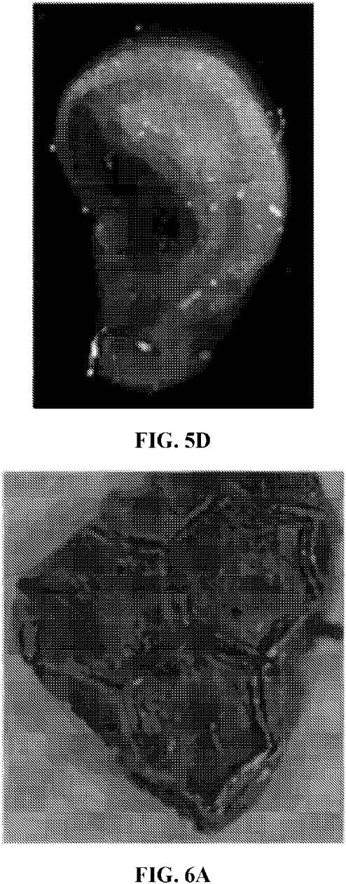

In another example, the materials described herein can be a genetically engineerable template for cell growth and regeneration. The bacteriophage hydrogel as disclosed herein can be used as a replacement or filler for cartilage in joints such as knees or for herniated or degenerated spinal discs. The bacteriophage as disclosed herein can be genetically engineered to promote cell binding and differentiation and thus help promote natural rejuvenation of the damaged site. In addition, the systems and methods described herein have been used to 3D print organ shapes with the bacteriophage hydrogel (see FIGS. 5A-5D). These organ scaffolds can form a template for specific cell growth and organ formation. 3D printed organs such as eye lenses, ear cochlea, nerves, heart valves and more can be used to replace damaged organs. The bacteriophage hydrogels can also be used as a biological glue to seal cuts and wounds. Another optional application is to use the bacteriophage hydrogels in cosmetics as a biodegradable injectable filler for cosmetic dermatology applications. This can be done using either a light-based or a copper catalyzed gelling process.

In another example, it can be possible to 3D print directly in the body. Both mechanisms of phage crosslinking: IR light or a reduction agent catalyzed by copper can be used for gelling within the body under spatial and temporal control using a pre-designed 3D structure.

A device for use in all these applications can be designed for each mechanism of crosslinking. For light based crosslinking a laparoscopic device in which the phage/photoinitiator mixture is released from a tube or needle at the same time as it is exposed to the right wavelength of light emitted from an LED. This would allow for immediate hydrogel formation as the liquid is extruded out of the tube or needle. In the second mechanism of crosslinking a mixture of phage with an excess of reduction agent would be extruded through a copper tubing resulting in crosslinking and hydrogel formation. This can be done via a laparoscopic device or injection with a syringe with a copper needle. Computer control over the XYZ axes of the tube or needle in either mechanism would allow for 3D printing within the body.

Other applications for inorganic material synthesis may include one or more of the following.

Different materials can be synthesized by using different mechanisms for material deposition in a specific manner on the bacteriophage surface. When the bacteriophage are crosslinked in a hydrogel, the material deposited on the virus will conform to the shape of the gel thus enabling macro-structural control over the material shape. Materials templated on bacteriophage hydrogels can make possible to 3D printing of materials that otherwise cannot be 3D printed. In addition, the micro to nano features of the bacteriophage hydrogel-templated material, elongated micro- to nano-scale rods, are unique to this synthesis method and can have advantages in the design and production of catalysts for heterogeneous catalysis, electrochemical catalysis and energy storage applications. Also materials that are relatively strong yet light can also be synthesized using these methods. Some of the materials that have been synthesized include:

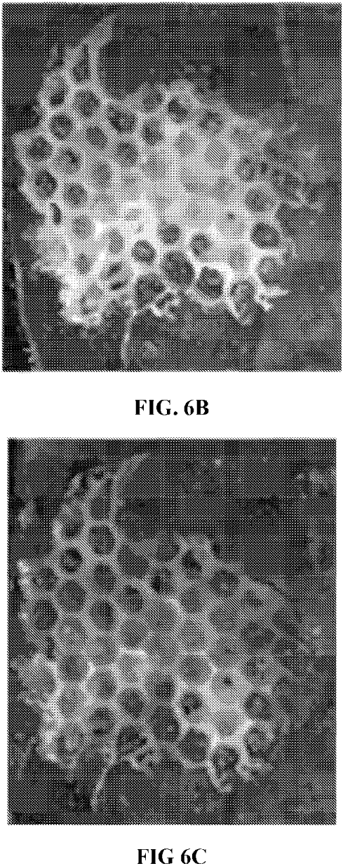

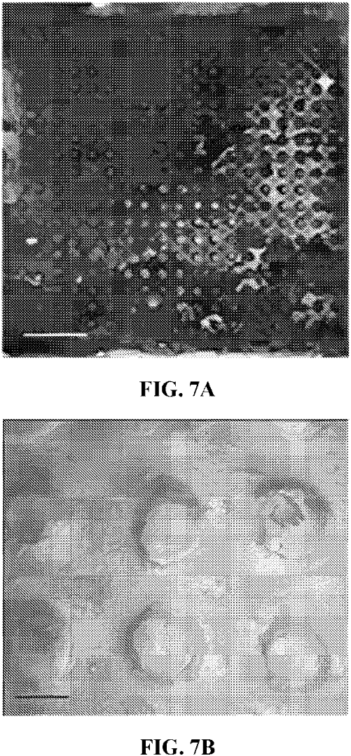

a) Perovskite materials - Lead perovskite has been synthesized in a stepwise synthesis in which lead carbonate was first deposited specifically on the phage surface and then turned into lead methylammonium halide by incubating in a solution of the desired methylammonium halide (see FIGS. 6A-6C). Perovskites can be used in solar cell and also as fluorescent sensors. See, for example, Holtus T, Helmbrecht L, Hendrikse HC, Baglai I, Meure S, Adhyaksa GW.P., Erik C. Garnett EC and Noorduin WL Shape-preserving transformation of carbonate minerals into lead halide perovskite semiconductors based on ion exchange/insertion reactions Nature Chemistry 2018, 10, 740-745, which is incorporated by reference in its entirety.

b) Hydroxyapatite - Soaking the bacteriophage hydrogel as disclosed herein in calcium chloride and then in sodium phosphate allows for deposition of hydroxyapatite on the phage surface. This can be useful for example in using 3D printing of the hydrogel to make synthetic teeth. Teeth are composed of an organic proteinaceous matrix covered in hydroxyapatite. The phage hydrogel can mimic the tooth organic matrix and can be engineered to bind ameloblast cells that can form enamel while the hydroxyapatite can be deposited to promote tooth formation and strengthen the template.

c) Vanadium nitride - A 3D printed hydrogel has been soaked in vanadium sulfate and then heated under an ammonia stream to form hard, conductive vanadium nitride aerogels that retain the original 3D printed shape. Vanadium nitride is a hard and stable material that 5 can be used as a structural light weight conductive material.

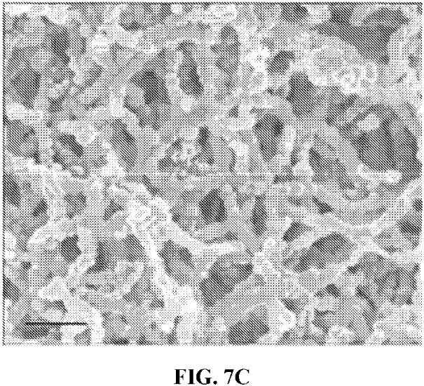

d) Copper or nickel aerogels - Activation of the phage surface with palladium followed by electroless deposition allows for copper or nickel deposition on the viral surface forming micro-sized rods with nano-scale width. See, for example, Ohmura JF, Burpo FJ, Lescott CJ, Ransil A, Yoon Y, Records WC and Belcher AM Highly adjustable 3D nano-architectures and chemistries via assembled 1D biological templates Nanoscale 2019, 11(3), 1091-1102, which is incorporated by reference in its entirety. It is possible to plate copper or nickel on 3D printed hydrogels allowing for control on the micro- to macro-scale and the ability to design structures needed for a particular application. For example, 3D printed bacteriophage based hydrogels can be used to make 3D printed electrodes for electrochemical catalysis (see FIGS. 7A-7C).

In certain circumstances, photopolymerization of virus particles is promising, owing to the homogeneity and high quality of virus-templated thin films prepared using this method.

### Diseases

The following are exemplary diseases/medical conditions that may be treated with the compositions and methods as disclosed herein: cancer (e.g., pancreatic ductal adenocarcinoma; pancreatic exocrine neoplasms; gastric adenocarcinoma; esophageal adenocarcinoma; squamous cell carcinoma of the gastrointestinal tract, head and neck, and genitourinary tract; hepatocellular carcinoma; pancreatic endocrine and neuroendocrine tumors; cholangiocarcinoma; glioblastoma; renal cell carcinoma; melanoma; small and large cell carcinoma of the lung; adenocarcinoma of the lung; squamous cell carcinoma of the lung; urothelial carcinoma and adenocarcinoma of the bladder; adenocarcinoma of the female reproductive system, including vaginal, cervical, endometrial, and ovarian cancer; testicular tumors, including seminomas and non-seminomas; prostate adenocarcinoma; breast carcinomas; or cutaneous T-cell lymphoma), tissue/organ regeneration (e.g., cartilage, discs, ocular tissue, ear drum or other small ear organs, valves, blood vessels; chronic wounds; pressure injuries; stress fractures; keloids; or burns), chronic inflammatory conditions (e.g., anti-inflammatory disease, autoimmune disease, immune modulation disorders, rheumatoid arthritis, osteoarthritis, inflammatory bowel disease, Crohn’s disease, ulcerative colitis, microscopic colitis, or collagenous colitis), diabetes (e.g. insulin release), aging (e.g., cosmetics, wrinkle fillers, lip augmentation), or pain (e.g., analgesia or anesthesia). The compositions as disclosed herein may also be used for delivery of antigens or mRNA’s for vaccines or for protection against radiotherapy. An injection (via endoscopy or interventional radiology guidance) to be injected around organs undergoing radiation therapy for cancer treatment is also disclosed herein.

In some embodiments, the hydrogel would act as a physical barrier to protect surrounding tissue/organs from radiation exposure and side effects and include proteins and peptides (growth factors and anti-inflammatory cytokines) to help in tissue healing and decrease of an inflammatory response in surrounding healthy tissue (e.g., injection around the prostate in radiation for prostate cancer could protect the adjacent colon and help to prevent radiation proctitis). In some embodiments, the hydrogel can also be engineered to slowly degrade so as to stay in long enough for tissue healing but will eventually be eliminated from the body.

In some embodiments, the compositions and methods as disclosed herein are administered to a patient systemically or locally, e.g., via intratumoral injection. In some embodiments, the compositions and methods as disclosed herein are administered to a patient alone or in combination with another therapy or therapeutic regimen, e.g., adjuvant therapy, e.g., co-administration with chemotherapy or immunotherapy. In some embodiments, the compositions and methods as disclosed herein are administered as an injection via endoscopy, catheters, or interventional radiology guidance (e.g., epidural needles) or standard needles. These delivery systems are equipped with either a copper needle at the end for catalyzing hydrogel formation using reducing agents or using a needle in conjunction with a light source to form hydrogels with photoinitiators.

### Methods

### Cysteine Phage Plasmid Construction

Oligopeptides representing a random octamer peptide fused to the N-terminus of the M13 bacteriophage pVIII major coat protein with two fixed cysteine residues at positions 3 and 6 from the N-terminal alanine were synthesized (Integrated DNA Technologies - IDT).

Random octamer peptide:

5′-AAGGCCGCTTTTGCGGGATCCNNMNNMNNGCAGTCCGGGCAMNNTGC
AGCGAAAGACAGC ATCGGAACGAGGGTAGCAACGGCTACAGAGGCTTT-
3′ (N = A, T, G or C;M = A or C) (SEQ ID NO: 4)

These random oligopeptides were annealed to a partially complementary oligopeptide -5′-TTAATGGAAACTTCCTCATGAAAAAGTCTTTAGTCCTCAAAGCCTCTGTAGCCGTT GCTACCCTCGTTCCGATGCTGTCTTTCGCTGCA-3′ (SEQ ID NO: 5), extended using Klenow Fragment DNA polymerase (NEB), and cut with BspHI and BamHI restriction enzymes. The resulting double stranded DNA strands were ligated into a modified M13KE plasmid cut with the same restriction enzymes and transformed into XL1-Blue electroporation-competent cells (Agilent Technologies catalog # 200228). The transformed cells were plated onto tetracycline IPTG/X-Gal plates with an excess of XL1-Blue cells for easy detection of phage plaques and grown overnight. Bacteriophage plaques were amplified for DNA purification and sequenced.

### Cysteine Phage Genome Construction for Expression of One or More Different Types of pVIII Proteins or for Expression of Proteins by Bacteriophage Infected E.coli

Part of a modified M13KE plasmid was PCR amplified using the following primers:

5′-CCTATTGGTTAAAAAATGAGCTGATTTAAC-3′ (SEQ ID NO: 6
)

and

5′-CCGAAATCGGCAAAATCCC-3′ (SEQ ID NO: 7)

An IL-2 gene fused to the N-terminus of pVIII one amino acid prior to the terminal alanine and containing an N-terminal pVIII leader peptide, a Shine-Dalgarno sequence, a LAC operator and a TAC promotor as well as a trpA terminator after the pVIII stop codons and 5′ and 3′ overlapping regions to the bacteriophage genome backbone amplified and described above was PCR amplified from a synthetically constructed gene using primers:

5′-GGGATTTTGCCGATTTCGG-3′ (SEQ ID NO: 8)

and

5′-GTTAAATCAGCTCATTTTTTAACCAATAGG-3′ (SEQ ID NO: 9
).

The two different PCR products were annealed and ligated using the Gibson assembly procedure with NEBuilder® HiFi DNA cloning master mix.

Additional genes can be engineered at the same site under the control of the same promotor or a different one, fused to pVIII or not fused to pVIII and/or with a similar or different terminator sequence.

Genome engineering of pIII is done as described in the NEB manual for Ph.D. Phage Display Libraries. Engineering pIX and pIII proteins is accomplished as described in Ghosh et al., ACS Synth Bio., 2012, 1(12), 576-582, which is incorporated by reference in its entirety.

### Cysteine Phage Amplification and Purification

Using a Wave 20/50 bioreactor in a 20 liter cell culture bag (CB0020L10-31), 10 liters of LB with 20 µg/mL tetracycline supplemented with a final concentration of 1 mM MgCl₂ were inoculated with a 1:100 dilution of an overnight culture of XL1-Blue cells. The bacteria were grown for 3 hours at 37° C., shaking at 250 rpm, to an O.D. of ~0.4 and then inoculated with a 100 ml of bacteria infected with cysteine-expressing bacteriophage as disclosed herein. Oxygen level and rocking speed were set to maximum. After an overnight amplification, the cells were filtered through two filters: 1.2 µm 0.4 m², and 0.8-0.45 µm 0.6 m². The supernatant containing the bacteriophage was concentrated from 10 liters to 500 mL using a tangential flow filtration system followed by a buffer exchange to PBS at a final volume of 850 mL. The solution was then spun down in one liter centrifuge bottles in an 8.10000 rotor (Beckman Coulter) at 8000 rpm for 30 minutes to remove leftover cell debris. The supernatant was collected in new centrifuge tubes, and a 20% PEG 8000/2.5 M NaCl solution was added at a 1:6 final ratio. The solution was then cooled overnight at 4° C. The precipitated bacteriophage were pelleted by centrifugation at 8000 rpm for 1.5-2 hours, and after discarding the supernatant, the pellet was re-suspended in 30 mL PBS. The re-suspended bacteriophage were centrifuged using a F0850 fixed angle rotor (Beckman Coulter) at 16500 rpm for 20 minutes to eliminate any leftover bacterial debris; a 20% PEG 8000/ 2.5 M NaCl solution was added to the supernatant at a 1:6 ratio and left overnight at 4° C. The bacteriophage were centrifuged at 14,000 rpm for 20 minutes and the resulting pellet was re-suspended in double distilled water or phosphate buffer saline pH 7.4 at a volume of ~15 mL, depending on the estimated yield, to obtain approximately 2-3×10¹⁴ phage/mL. Phage concentration was determined by absorbance at 269 nm and 320 nm.

Expression of bacteriophage genomes containing one or more additional pVIII genes and/or fusion proteins expressed as extensions of pIII and pIX are produced and purified in a similar manner.

### Crosslinking Cysteine Phage Into Hydrogels

### A) Light Based Crosslinking

A phage solution at a concentration of 1-3 phage/mL is mixed with a photoinitiator (addition of a norbornene containing crosslinker is optional) and illuminated with light for a period of 3 sec to 5 minutes.

Non-limiting examples of photoinitiators include:
1) lithium phenyl(2,4,6-trimethyl-benzoyl)phosphinate (TPO) (365-420 nm);
2) Eosin Y (~520 nm);
3) Methylene blue (requires TCEP) (665-710 nm);
4) Irgacure 2959 (UV ~280-365 nm); or
5) Bacteriochlorophyll derivatives (requires TCEP) (800 nm).

### B) Reduction Based Crosslinking

A phage solution at a concentration of 1-3 phage/mL is mixed with a reduction agent such as tris(2-carboxyethyl)phosphine (TCEP). The time for gelation depends on the ratio of reduction agent to phage disulfides. At a 1:1 ratio of TCEP to disulfides a hydrogel forms in about 30 seconds to 1 minute. At a ratio of 5:1 a hydrogel forms in a range of hours to overnight. This process can be accelerated by exposing the solution to a copper surface which causes gelation immediately upon contact. A copper needle can be connected to a syringe. When the bacteriophage/reducing agent solution in the syringe is injected through the copper needle, the bacteriophage crosslink and form a hydrogel that is extruded out the open end of the copper needle.

Other non-limiting examples of reduction agents can include:
1) Tris(hydroxypropyl)phosphine; or
2) Dithiothreitol (DTT) (requires copper to catalyze),

In one non-limiting example, large-area (1-10 cm²), thin-film, virus-templated nanofoams were also synthesized using a light-based photopolymerization technique on solutions of virus particles sandwiched between two substrates. Briefly, 6.45 cm² substrates were adhered to a large Petri dish using double-sided tape. Several substrate types were investigated, including Si wafers, glass, polycarbonate (PC), poly(methyl methacrylate) (PMMA), and titanium (Ti) foil, both with and without additional sputter-deposited, metallic (e.g., gold (Au) or copper (Cu)) layers on the surface. The most consistent adhesion between the hydrogel and substrate and the best final nanofoam quality were achieved by using rigid, flat, plasma-cleaned substrates (e.g., glass, Si wafers, PC, or PMMA) sputter-coated with a first 5-10 nm titanium adhesion layer and a second 50-100 nm gold layer as a bottom substrate. In addition to the bottom substrate, glass slides, previously cleaned with detergent and acetone in succession, were used as a top substrate. A hydrophobic coating was prepared on both sides of the glass slide to facilitate removal once the crosslinking reaction had finished. Three times in succession, 200 µL of Sigmacote was pipetted onto each side of the glass slide, wiped to facilitate an even application of the coating, and allowed to completely dry.

The photopolymerization reaction was mediated by lithium phenyl(2,4,6- trimethylbenzoyl)phosphinate (TPO), a Type I, water-soluble photoinitiator capable of initiating radical species in response to a ultraviolet/visible light stimulus. 6 wt% TPO stocks were prepared in 0.4 M K₂HPO₄ (balanced to pH 7.4). Next, 90 µL of 4.0 × 10¹³ pfu mL⁻¹ cysteine phage as disclosed herein and 0.5 wt% TPO (16.7 mM) was mixed and pipetted onto the gold-coated bottom substrate. A glass slide with the hydrophobic coating was then carefully placed over the droplet, sandwiching the phage solution between the hydrophobic top glass slide and the gold-coated bottom substrate. To crosslink the virus solution, the entire sample was irradiated with 365 nm light (ca. 5 mW cm⁻², UVP 95-0021-12 Model UVCL- 25 Compact Split Tube UV lamp, 115 V) for 60 seconds. Ultrapure water was slowly added to the container to completely submerge the bottom substrate, the virus hydrogel, and the glass slide on top. Following this, the top glass slide was slowly and smoothly drawn off in a direction parallel to the substrate until clear of the virus hydrogel. The best quality hydrogels and resulting nanofoams were achieved when the removal of the glass slide was accomplished with minimal vertical motion (perpendicular to the substrate). Each hydrogel was dialyzed in ultrapure water for 12-18 hours.

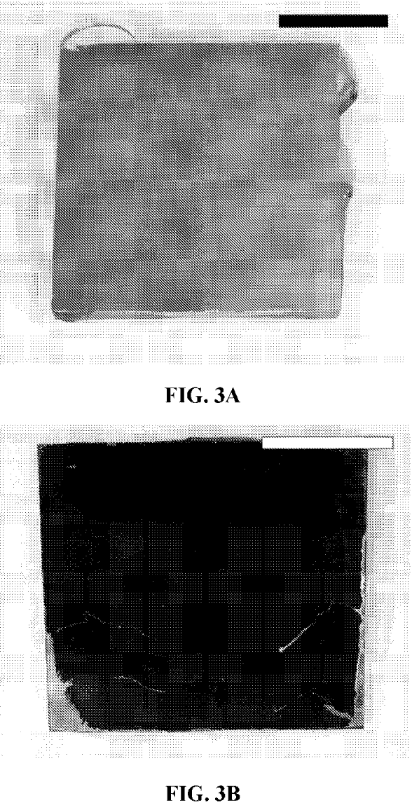

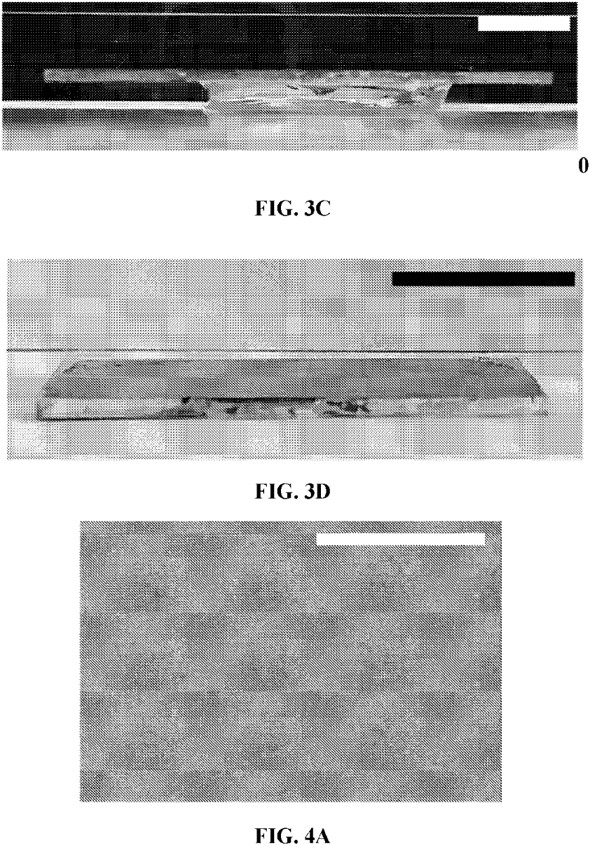

Based on the shortcomings of the first two methodologies, a third technique was developed to satisfy the following design constraints: (1) simultaneous exposure of the virus solution to a crosslinking stimulus, (2) greater control over hydrogel thickness, and (3) more scalable handling steps (e.g. right-side-up substrates). This technique makes use of a photopolymerization system. Specifically, a new virus clone was engineered to display cysteine residues on all of the pVIII coat protein (“cysteine phage”). Upon mixing the clone with a photoinitiator and irradiating with visible light, the thiol groups displayed by virus particles react to form covalent bonds and photopolymerize the mixture into a hydrogel (FIGS. 3A-3D). The process is safer, as it relies only on a light-based reaction between virus particles without any additional toxic crosslinkers. Additionally, using light as the stimulus instead of diffusing chemical species initiates the crosslinking chemistry simultaneously through the entire volume of the solution containing virus particles. Successful gelation occurred in samples with bacteriophage concentrations as low as 2 × 10¹³ pfu mL⁻¹ and with irradiation times as short as 3 seconds.

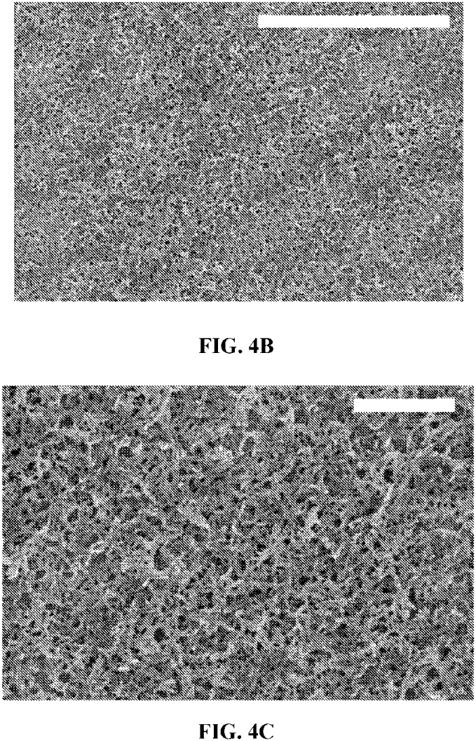

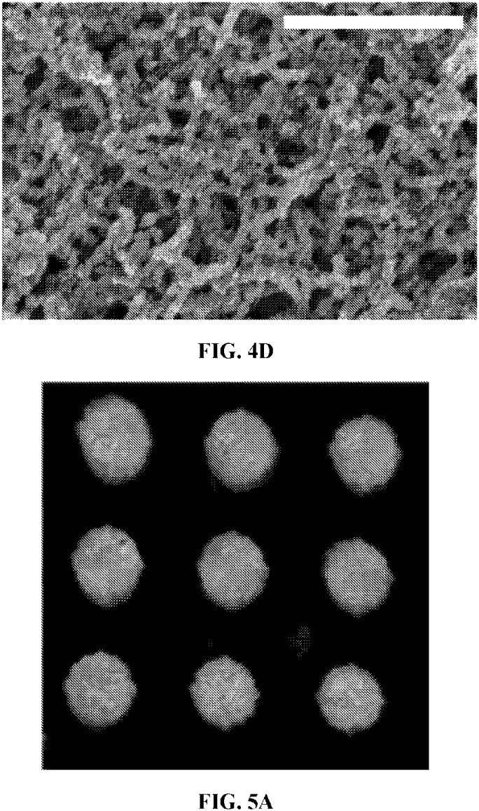

With the rapid gelation times, the shape of the virus solution was essentially “frozen” in place by the crosslinking reaction. Normal liquid droplets of bacteriophage as disclosed herein on a substrate, take a dome shape defined by the contact angle at the triple phase boundary between the air, solid substrate, and liquid, and were thus crosslinked into a hydrogel with a nonhomogeneous thickness. To promote heterogeneity and standardize the thickness, a further change was made to the methodology to sandwich the virus solution using a glass slide as a top substrate (FIG. 3C). Upon careful removal of the slide (previously coated with a hydrophobic surface layer), very thin (<1 mm) virus hydrogels could be reproducibly synthesized. The weight of the glass slide also spread the virus solution to cover the entire surface area of the substrate, ensuring flat and even coverage of the nanofoam after the subsequent synthesis steps (FIGS. 3B and 3D). SEM imaging of hydrogels metallized with copper reveal a homogeneous network morphology (FIGS. 4A-4D).

Other embodiments are within the scope of the following claims.

What is claimed is:

1. A genetically engineered bacteriophage comprising a plurality of peptides expressed at a surface of the bacteriophage, wherein each peptide comprises two or more cysteine residues.

2. The genetically engineered bacteriophage of claim 1, wherein the two or more cysteine residues form intra-peptide disulfide bonds within each peptide.

3. The genetically engineered bacteriophage of claim 1, wherein the plurality of peptides are displayed proximate to the N terminus of a plurality of p VIII major coat proteins.

4. The genetically engineered bacteriophage of claim 1, wherein each peptide further comprises a pVIII major coat protein.

5. The genetically engineered bacteriophage of claim 1, wherein each peptide comprises a CX(X)ₙC (SEQ ID NO: 1) motif, wherein n is 1, 2, 3, 4, 5, 6, 7, or 8.

6. The genetically engineered bacteriophage of claim 5, wherein each peptide comprises a CX(X)ₙC (SEQ ID NO: 1) motif, wherein n is 1.

7. The genetically engineered bacteriophage of claim 6, wherein the CXXC (SEQ ID NO: 1) motif is included in a XCPDCXXX (SEQ ID NO: 2) sequence.

8. The genetically engineered bacteriophage of claim 5, wherein each X is an amino acid residue selected from the group consisting of alanine (Ala), arginine (Arg), asparagine (Asn), aspartic acid (Asp), glutamine (Gln), glutamic acid (Glu), glycine (Gly), histidine (His), isoleucine (Ile), leucine (Leu), lysine (Lys), methionine (Met), phenylalanine (Phe), proline (Pro), pyrrolysine, selenocysteine, serine (Ser), threonine (Thr), tryptophan (Trp), tyrosine (Tyr), valine (Val), and a synthetic amino acid.

9. The genetically engineered bacteriophage of claim 1, wherein the bacteriophage is an M13, fd, f1, or ZJ/2 (Ff type) filamentous bacteriophage.

10. The genetically engineered bacteriophage of claim 1, wherein each peptide is glycosylated.

11. The genetically engineered bacteriophage of claim 1, wherein each peptide further comprises at least one protease-cleavable amino acid sequence distal to the two or more cysteine residues.

12. The genetically engineered bacteriophage of claim 1, wherein the peptides in the plurality of peptides are structurally substantially the same or have a similar function.

13. The genetically engineered bacteriophage of claim 1, wherein the peptides in the plurality of peptides include two or more structurally and/or functionally distinct populations of peptides.

14. The genetically engineered bacteriophage of claim 13, wherein the two or more structurally and/or functionally distinct populations of peptides function in tandem, sequentially, or in a cascade.

15. The genetically engineered bacteriophage of claim 1, wherein the bacteriophage further comprises at least one of a targeting moiety, an antibody, an antibody fragment, a bi-specific T-cell engager, an affibody, a nanobody, a cell penetrating peptide, a cytokine, a growth factor, a DNA repair enzyme, an opioid receptor-binding peptide, a protease, or a hormone.

16. The genetically engineered bacteriophage of claim 15, wherein:
(a) the at least one antibody is anti-PD-1, anti-PD-L1, or anti-CTLA4;
(b) the at least one antibody fragment is a single-chain variable antibody fragment;
(c) the at least one cytokine is IL-2, IL-7, IL-18, or IL-27;
(d) the at least one growth factor is IGF, NGF, GDNF, FGF, VEGF, TGF-alpha fragment, TGF-beta fragment, PDGF, or macrophage activator;
(e) the at least one DNA repair enzyme is endonuclease V;
(f) the at least one opioid receptor-binding peptide is enkephalin or substance P; and/or
(g) the at least one hormone is insulin, glucagon, ghrelin, angiotensin, or thyroid-stimulating hormone (TSH).

17. The genetically engineered bacteriophage of claim 1, wherein the bacteriophage further comprises at least one of a biotinylation protein, an antibiotic resistance gene, a bioluminescent protein, a fluorescent protein, or a chemiluminescent protein.

18. The genetically engineered bacteriophage of claim 17, wherein:
a) the biotinylation protein is BirA;
b) the bioluminescent protein is aequorin, firefly luciferase, Renilla luciferase, red luciferase, or nanoluciferase;
c) the fluorescent protein is EGFP, EYFP, ECFP, superfolder GFP, dsRed, mCherry, mOrange, mOrange2, mRaspberry, mTangerine, mApple, mRuby, mPlum, mKate1, mKate2, mKO2, mNeptune, mNeptune681, mNeptune684, mTurquoise, TagBFP, TagRFP675, azurite, EBFP2, mKalama1, iRFP682, iRFP713, iRFP720, miRFP703, miRFP670, miRFP670nano, miRFP682, miRFP702, miRFP703, miRFP709, miRFP713, miRFP720, iBlueberry, Wi-Phy, or mIFP; and/or
d) the chemiluminescent protein is β-galactosidase, alkaline phosphatase, or horseradish peroxidase (HRP).

19. The genetically engineered bacteriophage of claim 1, wherein the bacteriophage further comprises at least one different plurality of peptides expressed at the surface of the bacteriophage.

20. The genetically engineered bacteriophage of claim 19, wherein the at least one different plurality of peptides is displayed:
(a) proximate to the N terminus of a plurality of pIII minor coat proteins;
(b) proximate to the N terminus of a plurality of pIX minor coat proteins;
(c) proximate to the N terminus of a plurality of pVI minor coat proteins; and/or
(d) proximate to the N terminus of a plurality of pVII minor coat proteins.

21. The genetically engineered bacteriophage of claim 20, wherein the at least one different plurality of peptides displayed proximate to the N terminus of a plurality of a pIII, pIX, pVI, or pVII minor coat protein is substantially the same as, or has a similar function to, the at least one different plurality of peptides displayed proximate to the N terminus of a plurality of a different pIII, pIX, pVI, or pVII minor coat protein.

22. The genetically engineered bacteriophage of claim 20, wherein the at least one different plurality of peptides displayed proximate to the N terminus of a plurality of pIII minor coat proteins is substantially the same as or has a similar function to the at least one different plurality of peptides displayed proximate to the N terminus of a plurality of pIX, pVI, and/or pVII minor coat proteins.

23. The genetically engineered bacteriophage of claim 20, wherein the at least one different plurality of peptides displayed proximate to the N terminus of a plurality of a pIII, pIX, pVI, or pVII minor coat protein is different as or has a different function from at least one protein or peptide displayed proximate to the N terminus of a plurality of a different pIII, pIX, pVI, or pVII minor coat protein.

24. The genetically engineered bacteriophage of claim 20, wherein the at least one different plurality of peptides displayed proximate to the N terminus of a plurality of pIII minor coat proteins is different or has a different function from the at least one different plurality of peptides displayed proximate to the N terminus of a plurality of pIX, pVI, and/or pVII minor coat proteins.

25. The genetically engineered bacteriophage of claim 23, wherein the two or more structurally and/or functionally distinct populations of peptides function in tandem, sequentially, or in a cascade.

26-36. (canceled)

37. A method of forming a solid material, the method comprising:
(a) providing a plurality of genetically engineered bacteriophage, wherein each genetically engineered bacteriophage comprises a plurality of peptides expressed at a surface of the bacteriophage, wherein each peptide comprises two or more cysteine residues; and
(b) crosslinking the plurality of the genetically engineered bacteriophage to produce the solid material.

38-61. (canceled)

62. A method of making a genetically engineered bacteriophage genome construct, the method comprising:
(a) contacting a bacteriophage genome with restriction enzymes to produce a cleaved linear bacteriophage genome;
(b) contacting a heterologous nucleic acid with a second set of restriction enzymes to produce a cleaved heterologous nucleic acid, wherein the heterologous nucleic acid encodes a peptide having two or more cysteine residues; and
(c) ligating the cleaved linear bacteriophage genome with the cleaved heterologous nucleic acid in the presence of a ligase enzyme to produce a genetically engineered bacteriophage genome construct.

63. A method of making a genetically engineered bacteriophage genome construct, the method comprising:
(a) contacting a bacteriophage genome with a DNA polymerase enzyme in a polymerase chain reaction (PCR) to produce an extended bacteriophage genome; and
(b) ligating the extended bacteriophage genome with a heterologous nucleic acid in the presence of a ligase enzyme to produce a genetically engineered bacteriophage genome construct, wherein the heterologous nucleic acid encodes a peptide having two or more cysteine residues;
(c) optionally, prior to step (a), contacting a bacteriophage genome with restriction enzymes to produce a cleaved linear bacteriophage genome is performed.

64-66. (canceled)

67. A method of making a genetically engineered bacteriophage genome construct comprising:
(a) contacting a bacteriophage with DNA polymerase enzyme in a polymerase chain reaction (PCR), wherein the enzyme amplifies a part of the bacteriophage;
(b-1) contacting a heterologous nucleic acid, the heterologous nucleic acid encoding a peptide having two or more cysteine residues, with DNA polymerase enzyme in a different PCR, wherein the enzyme amplifies the heterologous nucleic acid, or
(b-2) contacting two complementary heterologous nucleic acids, wherein one of the heterologous nucleic acids encodes a peptide having two or more cysteine residues, with DNA polymerase enzyme in a different PCR, wherein the enzyme amplifies and anneals the two complementary nucleic acids; and
(c) ligating the amplified bacteriophage with the amplified heterologous nucleic acid in the presence of an exonuclease, a DNA polymerase, and a DNA ligase to produce a genetically engineered bacteriophage genome construct.

68-93. (canceled)

94. A genetically engineered bacteriophage comprising:
(i) a first plurality of peptides expressed at a surface of the bacteriophage, wherein each peptide comprises two or more cysteine residues and a pVIII major coat protein; and
(ii-1) a second plurality of fusion peptides expressed at a surface of the bacteriophage, wherein the second plurality of fusion peptides comprises a pVIII major coat protein or a pIII, pVI, pVII, or pIX minor coat protein fused to a distinct moiety designed for a specific purpose; or
(ii-2) a second plurality of peptides, wherein the second plurality of peptides comprises peptides that are expressed and/or secreted by a bacterial host cell.

95-105. (canceled)

106. A method of making a genetically engineered bacteriophage, the method comprising expressing
(i) a first plurality of peptides expressed at a surface of the bacteriophage, wherein each peptide comprises two or more cysteine residues and a pVIII major coat protein; and
(ii-1) a second plurality of peptides expressed at a surface of the bacteriophage, wherein the second plurality of peptides comprises a pVIII major coat protein or a pIII, pVI, pVII, or pIX minor coat protein fused to a distinct moiety designed for a specific purpose; or
(ii-2) a second plurality of peptides comprising peptides that are expressed and/or secreted by a bacterial host cell when the bacteriophage genome construct is propagated therein.

107. A method of making a genetically engineered bacteriophage genome construct, the method comprising:
(a) contacting a bacteriophage genome with restriction enzymes to produce a cleaved linear bacteriophage genome;
(b) contacting a first heterologous nucleic acid with a second set of restriction enzymes to produce a first cleaved heterologous nucleic acid, wherein the first heterologous nucleic acid encodes a peptide having two or more cysteine residues and a pVIII major coat protein;
(c) contacting a second heterologous nucleic acid with a third set of restriction enzymes to produce a second heterologous nucleic acid, wherein the second heterologous nucleic acid is selected from the group consisting of (i) and (ii), wherein the second heterologous nucleic acid;
(i) encodes a pVIII major coat protein or a pIII, pVI, pVII, or pIX minor coat protein fused to a distinct moiety designed for a specific purpose; and
(ii) encodes a plurality of peptides expressed and/or secreted by a bacterial host cell when the bacteriophage genome construct is propagated therein; and
(d) ligating the cleaved linear bacteriophage genome with the first and second cleaved heterologous nucleic acids in the presence of a ligase enzyme to produce a genetically engineered bacteriophage genome construct.

108. A method of making a genetically engineered bacteriophage genome construct, the method comprising:
(a) contacting a bacteriophage genome with a DNA polymerase enzyme in a polymerase chain reaction (PCR) to produce an extended bacteriophage genome; and
(b) ligating the extended bacteriophage genome with a first and second heterologous nucleic acid in the presence of a ligase enzyme to produce a genetically engineered bacteriophage genome construct;
(c) optionally, prior to step (a), contacting a bacteriophage genome with restriction enzymes to produce a cleaved linear bacteriophage genome;
wherein the first heterologous nucleic acid encodes a peptide having two or more cysteine residues and a pVIII major coat protein, and
wherein the second heterologous nucleic acid (i) encodes a pVIII major coat protein or a pIII, pVI, pVII, or pIX minor coat protein fused to a distinct moiety designed for a specific purpose or (ii) encodes a plurality of peptides expressed and/or secreted by a bacterial host cell when the bacteriophage genome construct is propagated therein.

109-128. (canceled)
